SEQUENCE LISTING (4 sequences)

SEQ ID NO: 1 - DNA, 20 bp, Artificial sequence
  Single strand DNA oligonucleotide
ccaccttaaaatctgcaggc

SEQ ID NO: 2 - DNA, 20 bp, Artificial sequence
  Single strand DNA oligonucleotide
gtgcatgaaggacagcctct

SEQ ID NO: 3 - DNA, 20 bp, Artificial sequence
  Single strand DNA oligonucleotide
cgggatgtctttttcttcca

SEQ ID NO: 4 - DNA, 20 bp, Artificial sequence
  Single strand DNA oligonucleotide
agcagcttcaggagccatag

(19) United States
(12) Patent Application Publication
(10) Pub. No.: US 2010/0145131 A1
(43) Pub. Date: Jun. 10, 2010

(54) METHODS AND KITS FOR PREDICTING CANCER METASTASIS

(76) Inventors: Helena Grinberg-Rashi, RaAnana (IL); Gideon Rechavi, Tel-Aviv (IL); Marina Perelman, Mevasseret Zion (IL); Shai Izraeli, Modiln (IL)

Correspondence Address: MARTIN D. MOYNIHAN d/b/a PRTSI, INC., P.O. BOX 16446, ARLINGTON, VA, 22215, US

(21) Appl. No.: 12/451,184

(22) PCT Filed: May 1, 2008
(86) PCT No.: PCT/IL08/00597

§ 371 Date: Feb. 25, 2010

Related U.S. Application Data
(60) Provisional application No. 60/924,147, filed on May 1, 2007.

(51) Int. Cl. A61M 36/06; C12Q 1/02; C12Q 1/48; C12Q 1/68

(52) U.S. Cl. 600/4; 435/29; 435/18; 435/6

(57) ABSTRACT

A method of predicting CNS metastasis of a non-neuronal cancer in a subject is disclosed. The method comprises determining a level and/or activity of N-cadherin (CDH2), in a sample of the subject wherein an increase in the CDH2 with respect to an unaffected sample is indicative of the CNS metastasis of the non-neural cancer. The method further comprises determining a level and/or activity or kinesin family member C1 (KIFC1) and/or Fetal Alzheimer Antigen (FALZ1) in the sample wherein an increase in KIFC1 and a decrease in FALZ with respect to an unaffected sample is further indicative of the CNS metastasis of the non-neural cancer. The method may be used for selection of a treatment regimen. In addition, kits for prediction CNS metastasis are disclosed.

### FIELD AND BACKGROUND OF THE INVENTION

The present invention, in some embodiments thereof, relates to methods and kits for predicting cancer metastasis and more particularly to brain metastasis.

Brain metastases are a serious complication of a number of cancers. They are most commonly associated with both small and non-small cell lung cancers (SCLC and NSCLC) (50-60%), followed by breast cancer (15-20%), melanoma (5-10%), and colon cancer (4-6%).

Lung cancer is the leading cause of cancer death worldwide. Between 75% and 85% of patients with primary lung malignancy have NSCLC. Staging is based on histopathology and extent of disease at presentation, but the heterogeneity of lung cancer patients with respect to outcome and treatment response suggests that additional sub-classification using molecular parameters is needed. While the brain is one of the major sites of relapse in NSCLC it is currently unclear which patient will develop this complication. Recent studies using microarray technology have shown a correlation between gene expression patterns in NSCLC and patient survival. None of those studies, however, specifically addressed the issue of brain metastases. Studies addressing that question [Arnold S M, et al., Clin Cancer Res 5:4028-33, 1999; D'Amico T A, et al., Ann Thorac Surg 72:1144-8, 2001; Kinch M S, et al., Clin Cancer Res 9:613-8, 2003] suggested that brain metastasis was related to an increased “malignant phenotype” manifested by expression of mutated P53 and Ki67 (a proliferation marker), coupled with expression of proteins mediating cell adhesion.

Prophylactic CNS directed therapy is a standard therapy in childhood leukemia and has recently proven to be beneficial in patients with SCLC. The high incidence of brain metastases in NSCLC has led to the suggestion of offering prophylactic CNS irradiation to these patients as well [Pottgen et al., J Clin Oncol 25:4987-92, 2007]. Accordingly, identification of patients at high risk for brain metastasis may enable better selection of those likely to benefit from prophylactic therapy to the CNS.

Additional background art includes Qi J, et al., Mol Biol Cell, 2005; Asano K, et al. J Neurooncol 70:3-15, 2004; Hulit J, et al. Cancer Res 67:3106-16, 2007; Hazan R B, et al., J Cell Biol 148:779-90, 2000; Ramaswamy S, et al. Nat Genet 33:49-54, 2003; Erez et al., Oncogene 23:5371-7, 2004; Corson T W et al., Clin Cancer Res 13:3229-34, 2007; Haruki N, et al. Cancer Lett 162:201-5, 2001; Takahashi T, et al. Oncogene 18:4295-300, 1999.

### SUMMARY OF THE INVENTION

According to an aspect of some embodiments of the present invention there is provided a method of predicting central nervous system (CNS) metastasis of a non-neuronal cancer in a subject, the method comprising determining a level and/or activity of N-cadherin (CDH2), in a sample of the subject wherein an increase in the CDH2 with respect to an unaffected sample is indicative of the CNS metastasis of the non-neural cancer.

According to another aspect of some embodiments of the present invention there is provided a method of treating a subject having a non-neuronal cancer, the method comprising:

(a) determining a level and/or activity of N-cadherin (CDH2), in a sample of the subject; and

(b) determining a treatment regimen based on the level and/or activity of the CDH2.

According to another aspect of some embodiments of the present invention there is provided a kit for predicting CNS metastasis of a non-neuronal cancer in a subject, the kit comprising a packaging material which comprises at least one agent for specifically determining a level and/or activity of no more than one hundred markers, wherein at least one of the one hundred markers is N-cadherin (CDH2).

According to some embodiments of the invention, the method further comprises determining a level and/or activity or kinesin family member C1 (KIFC1) and/or Fetal Alzheimer Antigen (FALZ1) in the sample of the subject wherein an increase in the KIFC1 and a decrease in the FALZ with respect to an unaffected sample is further indicative of the CNS metastasis of the non-neural cancer.

According to some embodiments of the invention, the non-neuronal cancer is selected from the group consisting of non-small cell lung cancer, breast cancer and colon cancer.

According to some embodiments of the invention, the non-neuronal cancer is non-small cell lung cancer.

According to some embodiments of the invention, the method further comprises determining a level and or activity of at least one additional marker involved in cell proliferation and mitosis, wherein an increase in the additional marker is further indicative of CNS metastasis of the neuronal cancer.

According to some embodiments of the invention, the at least one additional marker is selected from the group consisting of KIFC1 (kinesin family member C1), KIF2C (kinesin family member 2C), KIF14 (kinesin family member 14), CCNB2 (cyclin B2), SIL (SCL-TAL1 interrupting locus) and TNPO1 (transportin I).

According to some embodiments of the invention, the treatment regimen is selected from the group consisting of CNS radiotherapy, intrathecal chemotherapy and intravenous chemotherapy.

According to some embodiments of the invention, the kit further comprises agents for specifically determining a level and/or activity of at least one marker selected from the group consisting of kinesin family member C1 (KIFC1) and Fetal Alzheimer Antigen (FALZ1).

According to some embodiments of the invention, the kit further comprises agents for specifically determining a level and/or activity of at least one marker selected from the group consisting of KIF2C (kinesin family member 2C), KIF14 (kinesin family member 14), CCNB2 (cyclin B2), SIL (SCL-TAL1 interrupting locus) and TNPO1 (transportin I).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an illustration of an exemplary CDH2 and SIL matrix on 96-well plates which contained duplicates for 10 lung tumor samples, a sample of H1299, and a negative control (wells without cDNA).

FIGS. 2A-F are graphs illustrating the distribution of quantitative real time RT-PCR values prior to and following normalization for the various genes: FIG. 2A—CDH2, FIG. 2B—ADAMS, FIG. 2C—SIL, FIG. 2D—TNPO1, FIG. 2E—LMNB1, FIG. 2F—CCNB2, FIG. 2G—KIFC1, FIG. 2H—KIF2C, FIG. 2I—KIF14, FIG. 2J—FALZ, FIG. 2K—SGNE1, FIG. 2L—SPP1. The upper panels represents the absolute values, the lower panels represents the values after normalization

FIG. 3 is a schematic illustration of the relative effect of each gene on the risk for brain metastasis based on its Wald score. Genes in the center had no significant effect while genes on the right had a positive effect and genes on the left had a negative effect. A score of 2.71 and above was considered significant.

FIGS. 4A-B are graphs illustrating Kaplan-Meier analysis for brain Metastases Free Survival of NSCLC patients. Patients were stratified by the brain metastasis score to three ranking groups low (1.00), medium (2.00), and high (3.00) on the basis of the three-gene expression model. FIG. 4A—Stage I-II disease p<0.02 log rank test. FIG. 4B—Stage III-IV disease P<0.02, log rank test.

FIGS. 5A-C are photographs of H&E stains and anti-CDH2 immunostains (×20). FIG. 5A—Positive control (mesothelioma); FIG. 5B—Primary lung squamous cell carcinoma negative for CDH2; FIG. 5C—squamous cell carcinoma positive for CDH2.

FIG. 6A is a graph and table illustrating CDH2 immunostaining in 107 NSCLC Patients. 60% of the tumor samples from patients that developed brain metastasis were positive for N-cadherin compared to only 29% of the tumor samples from patients that did not develop brain metastasis.

FIGS. 6B-C are graphs illustrating Kaplan-Meier analysis for brain Metastases Free Survival of NSCLC patients according to CDH2 Immunostaining. FIG. 6B—All 107 NSCLC samples analyzed by immuno-histochemistry. (p<0.022 log rank test) FIGS. 6C-63 independent samples which were not included in the RQ-PCR cohort (p<0.03, log rank test).

FIGS. 7A-E are photographs of anti-CDH2 immunostains (×20). FIG. 7A—positive control (mesothelioma), FIG. 7B—primary tumor with brain metastases, FIG. 7C—primary tumor without brain metastases, FIGS. 7D, E—brain metastases (in E normal brain strongly positive for N-Cadherin).

### DESCRIPTION OF SPECIFIC EMBODIMENTS OF THE INVENTION

The present invention, in some embodiments thereof, relates to methods and kits for predicting cancer metastasis and more particularly to cancer metastasis to the CNS.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Brain metastases affect 25% of patients with non small cell lung cancer (NSCLC). The present inventors hypothesized that the expression of genes in primary NSCLC tumors could predict brain metastasis and be used for identification of high risk patients who may benefit from prophylactic therapy to the central nervous system.

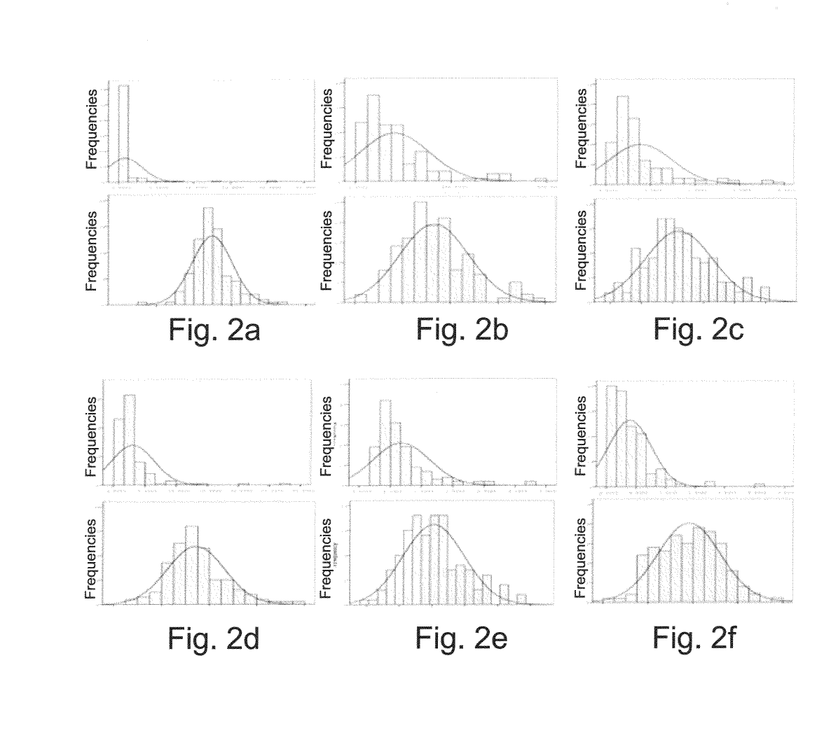

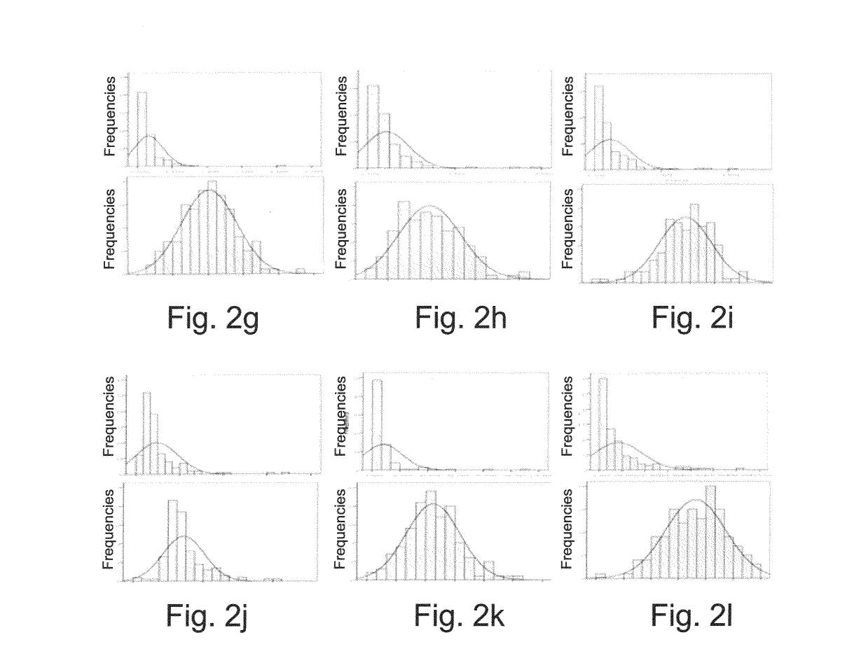

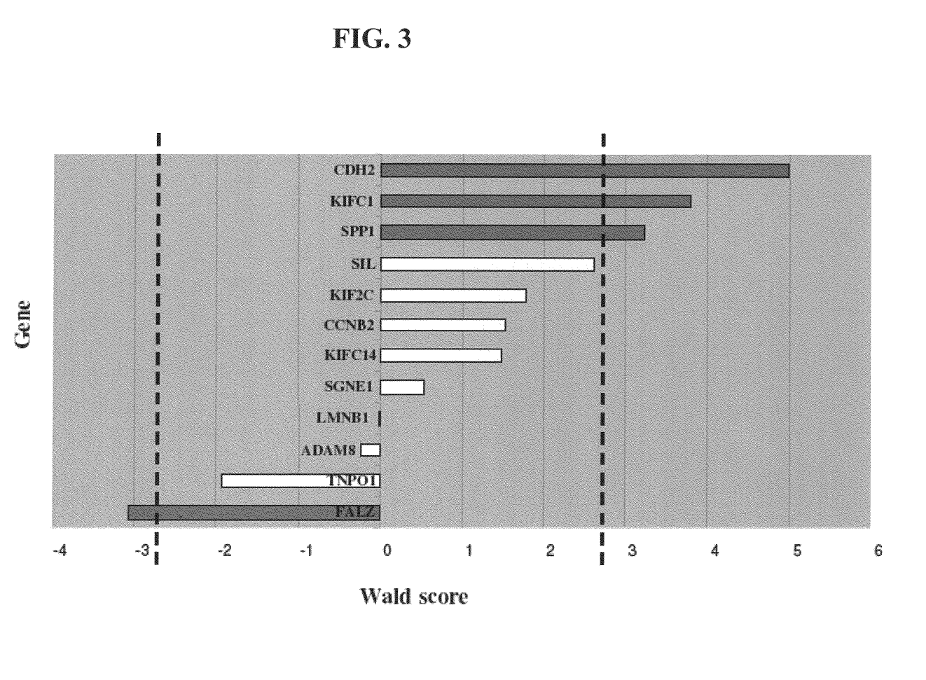

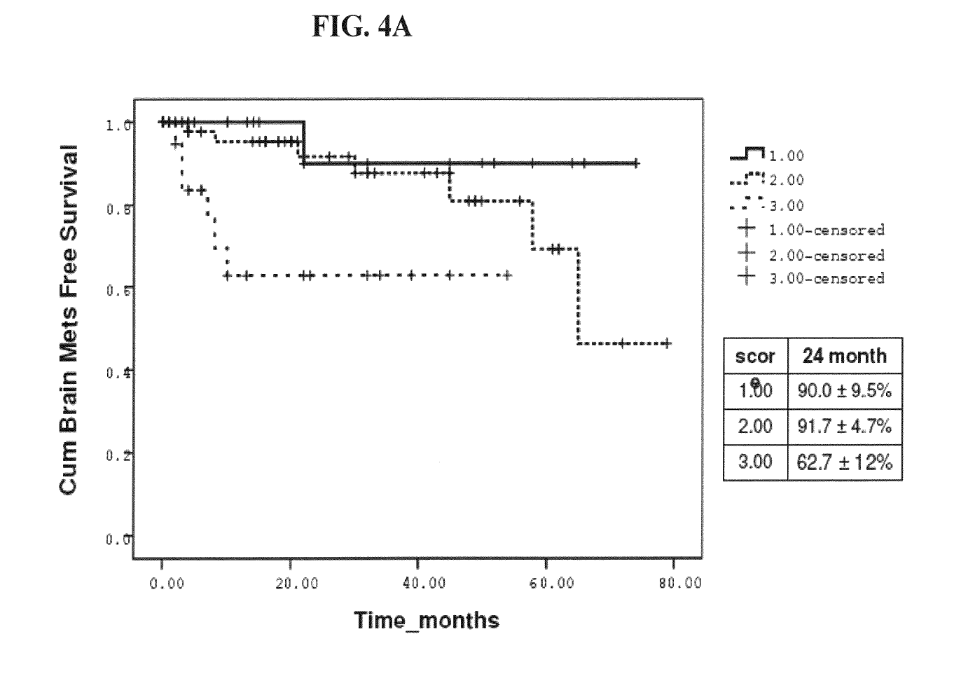

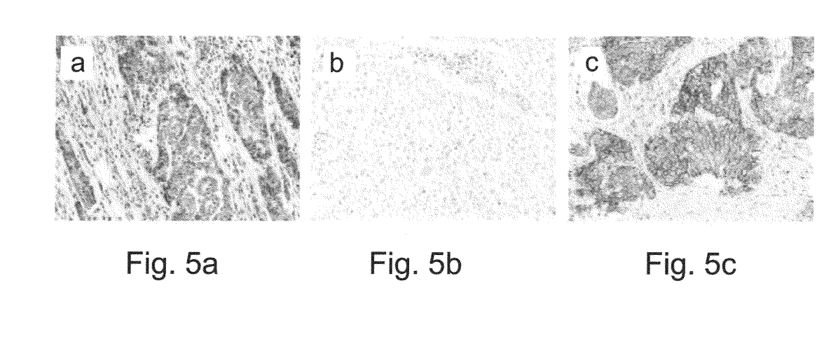

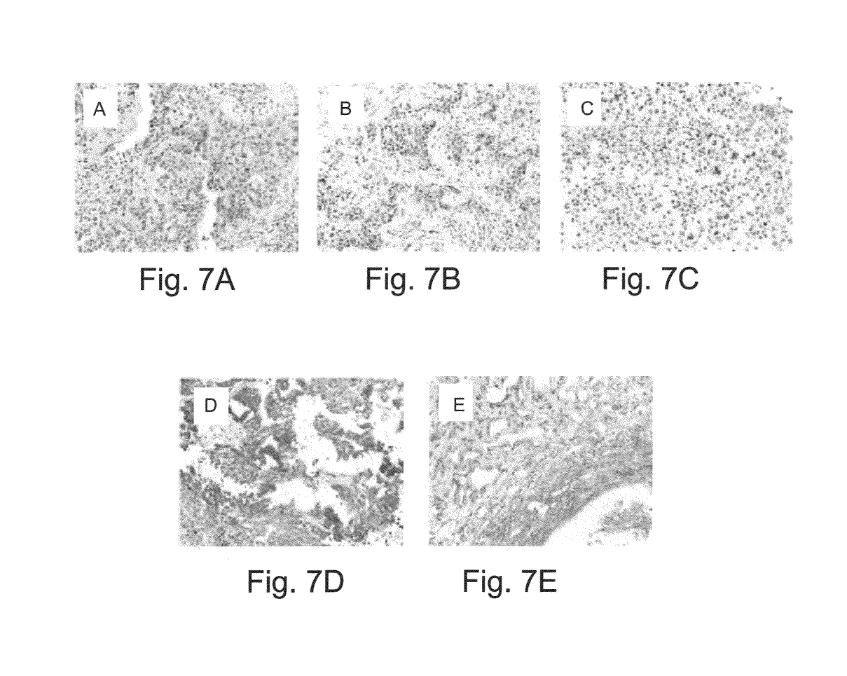

Whilst reducing the present invention to practice, the present inventors identified three genes, CDH2 (N-cadherin), KIFC1 and FALZ that were highly predictive of brain metastasis in early as well as advanced lung cancer. Cox regression analysis was used to analyze the correlation between gene expression (as measured by real time quantitative reverse transcriptase polymerase chain reaction) and the occurrence of brain metastasis (FIGS. 2A-F and FIG. 3). Immunohistochemistry on independent samples was used to verify the findings (FIGS. 5A-C and FIGS. 7A-E).

The probability of remaining brain metastasis free at two years after diagnosis for patients with stage I/II tumors and low score was 90.0±9.5% compared with 62.7±12% for patients with high score (p<0.01). In patients with more advanced lung cancer the brain metastasis free survival at 24 months was 89% for patients with low score compared with only 37% in patients with high score (P<0.02). Similarly immunohistochemical detection of N-cadherin in primary NSCLC also predicted brain metastasis.

Thus, according to one aspect of the present invention, there is provided a method of predicting CNS metastasis of a non-neuronal cancer in a subject, the method comprising determining a level and/or activity of N-cadherin (CDH2), in a sample of the subject wherein an increase in the CDH2 with respect to an unaffected sample is indicative of the CNS metastasis of the non-neural cancer.

The term “predicting” as used herein refers to determining the presence of brain metastasis either prior to the event of metastasis or following the event of metastasis i.e. diagnosing.

According to the prediction, a subject exhibiting the increase in CDH2 expression may be classified as being susceptible to CNS metastasis.

The term “diagnosing” as used herein refers to determining the presence of a CNS metastasis, classifying a CNS metastasis, determining a severity of CNS metastasis, monitoring CNS metastasis progression, forecasting an outcome of the CNS metastasis and/or prospects of recovery.

As used herein, the phrase “CNS metastasis” refers to the spread of a tumor, from one part of the body to the central nervous system (i.e. brain or spinal cord).

The phrase “non-neuronal cancer” as used herein, refers to a cancer of cells from a non-neuronal origin.

Exemplary non-neuronal cancers include, but are not limited to non-small cell lung cancer (NSCLC), small cell lung cancer (SCLC), breast cancer, mesotheliomas, melanoma, ovarian carcinoma, bladder cancer, renal cancer and colon cancer.

The cancer may be at any stage of any cell type including but not limited to adenocarcinoma and squamous cell carcinoma.

The subject may be an animal (e.g. mammal) or human diagnosed with a non-neuronal cancer.

Samples of the subject are typically derived from the site of the primary tumor, e.g. during a tumor biopsy.

Exemplary methods of biopsy include, but are not limited to bronchoscopic biopsy, needle biopsy, CT-guided needle biopsy, endoscopic biopsy, skin biopsy, open biopsy, mediastinoscopy and video-assisted thorascopic surgery.

Control samples to which the subject's samples are compared may be obtained from cancer patients with the same cancer type, preferably at the same stage, wherein the clinical outcome of the cancer is known not to comprise brain metastasis. It is preferable that the non-metastatic cancerous control sample come from a subject of the same species, age and from the same sub-population (e.g. smoker/nonsmoker). Alternatively, control data may be taken from databases and literature. It will be appreciated that the control sample may also be taken from the diseased subject at a particular time-point, prior to metastasis in order to analyze the progression of the disease.

As mentioned, the method of the present invention is affected by determining an expression or activity of N-cadherin, wherein an increase in CDH2 with respect to the unaffected sample is indicative of a CNS metastasis.

The term “N-cadherin” refers to the transmembrane, glycol-polypeptide, such as set forth by Genebank accession number NP_{—}001783 (the mRNA of which is as set forth in NM_{—}001792), transcribed from the genomic sequence NC_{—}000018.8 from positions 24011189 to 23784933.

Determining an expression of N-cadherin may be effected on the RNA or protein level as detailed below.

Methods of Detecting Expression of N-Cadherin on the RNA Level

Northern Blot analysis: This method involves the detection of a particular RNA i.e. N-cadherin RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Detection may be using autoradiography, colorimetric reaction or chemiluminescence. This method allows both quantitation of an amount of particular RNA molecules and determination of its identity by a relative position on the membrane which is indicative of a migration distance in the gel during electrophoresis.

RT-PCR analysis: This method uses PCR amplification of relatively rare RNAs molecules. First, RNA molecules are purified from the cells and converted into complementary DNA (cDNA) using a reverse transcriptase enzyme (such as an MMLV-RT) and primers such as, oligo dT, random hexamers or gene specific primers. Then by applying gene specific primers and Taq DNA polymerase, a PCR amplification reaction is carried out in a PCR machine. Those of skills in the art are capable of selecting the length and sequence of the gene specific primers and the PCR conditions (i.e., annealing temperatures, number of cycles and the like) which are suitable for detecting specific RNA molecules. It will be appreciated that a semi-quantitative RT-PCR reaction can be employed by adjusting the number of PCR cycles and comparing the amplification product to known controls. Exemplary primers that may be used to detect N-cadherin are set forth in SEQ ID NOs: 1 and 2.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Oligonucleotide microarray—In this method oligonucleotide probes capable of specifically hybridizing with the polynucleotides of the present invention are attached to a solid surface (e.g., a glass wafer). Each oligonucleotide probe is of approximately 20-25 nucleic acids in length. To detect the expression pattern of the polynucleotides of the present invention in a specific cell sample (e.g., blood cells), RNA is extracted from the cell sample using methods known in the art (using e.g., a TRIZOL solution, Gibco BRL, USA). Hybridization can take place using either labeled oligonucleotide probes (e.g., 5′-biotinylated probes) or labeled fragments of complementary DNA (cDNA) or RNA (cRNA). Briefly, double stranded cDNA is prepared from the RNA using reverse transcriptase (RT) (e.g., Superscript II RT), DNA ligase and DNA polymerase I, all according to manufacturer's instructions (Invitrogen Life Technologies, Frederick, Md., USA). To prepare labeled cRNA, the double stranded cDNA is subjected to an in vitro transcription reaction in the presence of biotinylated nucleotides using e.g., the BioArray High Yield RNA Transcript Labeling Kit (Enzo, Diagnostics, Affymetrix Santa Clara Calif.). For efficient hybridization the labeled cRNA can be fragmented by incubating the RNA in 40 mM Tris Acetate (pH 8.1), 100 mM potassium acetate and 30 mM magnesium acetate for 35 minutes at 94° C. Following hybridization, the microarray is washed and the hybridization signal is scanned using a confocal laser fluorescence scanner which measures fluorescence intensity emitted by the labeled cRNA bound to the probe arrays.

For example, in the Affymetrix microarray (Affymetrix®, Santa Clara, Calif.) each gene on the array is represented by a series of different oligonucleotide probes, of which, each probe pair consists of a perfect match oligonucleotide and a mismatch oligonucleotide. While the perfect match probe has a sequence exactly complimentary to the particular gene, thus enabling the measurement of the level of expression of the particular gene, the mismatch probe differs from the perfect match probe by a single base substitution at the center base position. The hybridization signal is scanned using the Agilent scanner, and the Microarray Suite software subtracts the non-specific signal resulting from the mismatch probe from the signal resulting from the perfect match probe.

Methods of Detecting N-Cadherin on the Protein Level

Determining expression of N-cadherin on the protein level is typically effected using an antibody capable of specifically interacting with N-cadherin. Exemplary antibodies capable of specifically interacting with N-cadherin are available from DakoCytomation, California, USA (Monoclonal mouse anti human CDH2, clone 6G11, cat number: M361301). Methods of detecting N-cadherin include immunoassays which include but are not limited to competitive and non-competitive assay systems using techniques such as Western blots, radioimmunoassays, ELISA (enzyme linked immunosorbent assay), “sandwich” immunoassays, and immunoprecipitation assays and immunohistochemical assays as detailed herein below.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

In situ activity assay: According to this method, a chromogenic substrate is applied on the cells containing an active enzyme and the enzyme catalyzes a reaction in which the substrate is decomposed to produce a chromogenic product visible by a light or a fluorescent microscope.

As mentioned, the method of the present invention may also be effected by measuring an activity of N-cadherin.

As used herein, the phrase “N-cadherin activity” refers to N-cadherin mediated cell aggregation, adhesion, migration and/or invasion. Such activities may be measured using a variety of different assay methods designed to measure, for example, cell migration, aggregation, adhesion and invasion.

According to one embodiment the N-cadherin activity is an N-cadherin/FGF-2 mediated signal transduction that can be assayed by measuring the activity and/or expression of components in the FGF-2 signal transduction pathway—see for example U.S. Patent Application No. 20030054985, incorporated herein by reference. Such assays include detection of N-cadherin/FGFR complexes, increased MMP-9 expression and/or activation of MAPK activity.

Assaying N-cadherin activity may be effected using a variety of different methods. For example, coaggregation assays may be used to measure an amount of N-cadherin. In such assays, single cell suspensions of cells are visualized to determine the extent of cell aggregation. In a specific embodiment of the invention, the cells may be labeled with a fluorescent dye prior to mixing, to facilitate visualization of aggregating cells.

Alternatively, measuring N-cadherin activity may be effected by analyzing adhesion of cells to endothelium. For example, human endothelium monolayers may be formed by plating HUVEC cells on gelatin coated cover slips. A cell sample is then added to the endothelium monolayers and incubated for a time sufficient to allow adhesion to the monolayer. The level of cell adhesion is measured.

In yet another embodiment of the invention, activation or suppression of matrix metalloproteinase-9 activity or MAPK activity can be measured as an indicator of N-cadherin levels. Levels of matrix metalloproteinase-9 can be measured using, for example, substrate gel electrophoresis (Zymography) as described in Nakajima et al. Nakajima I et al., 1995, Br. J. Cancer. 71:1039-1045). Levels of MAPK activity can be measured as described in U.S. Patent Application No. 20030054985. It will be appreciated that in order to increase predictability of a CNS metastasis other markers in the primary tumors may also be analyzed. Thus, for example, an expression or activity of kinesin family member C1 (KIFC1) may also be analyzed wherein an increase thereof with respect to an unaffected sample is further indicative of CNS metastasis of the non-neural cancer.

As used herein, the term “kinesin family member C1” refers to the tubulin binding polypeptide such as set forth by Genebank accession number NP_{—}002254, (the mRNA of which is as set forth in NM_{—}002263), transcribed from the genomic sequence NC_{—}000006.10 from positions 33467583 to 33485625.

According to another embodiment, an activity or expression of Fetal Alzheimer Antigen (FALZ1) may be analyzed as well as N-cadherin, wherein a decrease thereof with respect to an unaffected sample is further indicative of the CNS metastasis of the non-neural cancer.

As used herein, the term “FALZ” refers to neuronal transcriptional factor such as set forth by Genebank accession number NP_{—}004450 or NP_{—}872579, (the mRNA of which is set forth in NM_{—}004459), transcribed from the genomic sequence NC_{—}000017.9 from positions 63252242 to 3410956.

Although N-cadherin can be used individually whilst providing statistical significant diagnosis, the present inventors have shown that analysis of all three markers (i.e. N-cadherin, KIFC1 and FALZ1) in a particular sample allows for a very high prediction of CNS metastasis. Thus, if in a given sample, there is an increase in expression of N-cadherin and kinesin family member C1 and a decrease in expression of FALZ, it may be predicted with a high probability that the cancer will metastasize to the CNS.

It will be appreciated that the present invention contemplates that the change in expression of the markers of the present invention may be in the same cell or may be an overall change in a cell population.

Methods of analyzing expression of KIFC1 and FALZ1 are identical to those described for N-cadherin, as described herein above. Antibodies capable of specifically recognizing KIFC1 are commercially available—e.g. from ABR-affinity BioReagents (Cat. No. MA1-53105) or Bethyl Laboratories (Cat. No. A300-951A). Antibodies capable of specifically recognizing FALZ are also commercially available—e.g. from Novus Biologicals (Cat. No. NB100-41418) or Bethyl Laboratories (Cat. No. A300-973A). Exemplary primers that may be used to detect N-cadherin are set forth in SEQ ID NOs: 3 and 4.

Other markers that may also be analyzed in order to raise the accuracy of the prediction include cell proliferation and mitosis markers, wherein an increase in these marker are further indicative of CNS metastasis of the neuronal cancer.

Exemplary cell proliferation and mitosis markers that may be analyzed according to this aspect of the present invention include, KIF2C (kinesin family member 2C), KIF14 (kinesin family member 14), CCNB2 (cyclin B2), SIL (SCL-TAL1 interrupting locus) and TNPO1 (transportin I).

It will be appreciated that the tools necessary for detecting the CNS metastases markers of the present invention may be provided as a kit, such as an FDA-approved kit, which may contain one or more unit dosage form containing the active agent (e.g. antibody or probe) for detection of at least one marker of the present invention. According to one embodiment, the kit comprises active agents for detection of all three markers of the present invention. According to yet another embodiment, the kit comprises active agents for no more than five markers. According to yet another embodiment, the kit comprises active agents for no more than 10 markers. According to yet another embodiment, the kit comprises active agents for no more than 20 markers. According to yet another embodiment, the kit comprises active agents for no more than 50 markers. According to yet another embodiment, the kit comprises active agents for no more than one hundred markers.

The kit may be accompanied by instructions for administration. The kit may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration.

It will be appreciated that the method of the present invention may be affected together with other methods for diagnosing metastasis to order to improve the accuracy of the prediction. Thus, for example, imaging studies such as CT and/or MRI may be obtained to further diagnose the metastasis.

Since the method of the present invention may be used to identify patients at high risk for CNS metastasis, the present invention may also be used to determine a treatment regimen for such patients. Accordingly, patients found to be at high risk for CNS metastasis as determined using the method of the present invention may be treated with prophylactic therapy to the central nervous system.

Exemplary treatment regimes that may be used as prophylactic therapy to the CNS include, but are not limited to CNS radiotherapy, intrathecal chemotherapy and intravenous chemotherapy (e.g. with methotrexate).

Determination of the specific treatment regime will, of course, be dependent on the subject being treated, the severity of the affliction, the manner of administration and the judgment of the prescribing physician, etc.

It is expected that during the life of a patent maturing from this application many relevant prophylactic CNS therapies will be developed and the scope of the term treatment regimen is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Development of a Model Predicting the Risk for Brain Metastasis Based on Gene Expression Levels in Primary Tumors

Materials and Methods

Gene Expression Real Time RT PCR (RQ-PCR) Study

Patients and Samples: RNA was extracted from 230 consecutive frozen samples of lung tumor tissues, obtained from patients who underwent surgery, stored at pulmonary institute of the Sheba Medical Center. 190 of these samples yielded at least 5 μg high quality RNA. Review of the pathological records confirmed the diagnosis of NSCLC in 142 patients. Clinical data was obtained from patients' files including radiological and pathological records (Table 1, herein below).

TABLE 1

| Characteristic | RQ-PCR study | CDH2 IHC study |
|---|---|---|
| Number of cases: | | |
| With brain metastasis | 31 | 25 |
| Without brain metastasis | 111 | 82 |
| Age at diagnosis of | | |
| primary tumor- yr: | | |
| Mean ± SD | 64.95 ± 1.29 | 61.06 ± 1.22 |
| Sex - no. (%): | | |
| Male | 93 (65.5) | 76 (71.0) |
| Female | 49 (34.5) | 31 (29.0) |
| Cancer cell type - no. (%): | | |
| Adenocarcinoma | 66 (46.5) | 43 (40.2) |
| Squamous Cell Carcinoma | 61 (43) | 50 (46.7) |
| Other | 15 (10.5) | 14 (13.1) |
| Stage at diagnosis- no. (%): | | |
| I or II | 97 (68) | 76 (71) |
| III or IV | 45 (32) | 31 (29) |
| Follow up period*(months): | | |
| Median | 22 | 34 |
| Range | 1-81 | 1-72 |

*Follow up period was defined as the time from surgery to death or last visit in hospital.

Staging was determined according to the tumor, node metastasis system (TNM). The diagnosis of brain metastasis was based on CT or MRI records. The time from diagnosis of lung cancer until the date of brain imaging demonstrating brain metastasis was defined as “time to brain metastasis”. Follow up period was defined as the time from surgery to death or last visit in hospital.

Selection of Genes for RNA Quantification: Based on gene expression studies on Affymetrix U95 chips (containing 1265 probe sets), twelve genes belonging to three general functional categories were selected:

Cell proliferation and mitosis: KIFC1 (kinesin family member C1), KIF2C (kinesin family member 2C), KIF14 (kinesin family member 14), CCNB2 (cyclin B2), SIL (SCL-TAL1 interrupting locus), TNPO1 (transportin I), LMNB1 (Lamin B1).

Neuronal genes: CDH2 (N-cadherin), SGNE1 (Secretogranin V), FALZ (Fetal Alzheimer Antigen).

Genes coding extra-cellular matrix proteins: ADAM8 (A Disintegrin and Metalloprotease 8) and SPP1 (Osteopontin).

RNA processing and Real time PCR: Total RNA was isolated using Trizol (Carlsbad Calif., Invitrogen, USA). RNA isolated from a NSCLC cell line (H1299) served as a calibrating control. RQ-PCR was then performed on cDNA synthesized from the RNA using ABgene Reverse-iT™ 1^{st} Strand Synthesis Kit (ABgene, Surrey, UK). RQ-PCR assays were performed with the ABI Prism® 7900 sequence detection system using the SDS 2.2 software application. Taqman Gene Expression Assays were developed using two specific oligonucleotide primers and a unique Taqman MBG probe for the fluorescently marked target sequence (as detailed in Table 2, herein below).

TABLE 2

| | | NCBI RefSeq | TaqMan Gene |
|---|---|---|---|
| Symbol | Gene name | number | Expression Assay ID |
| KIFC1 | kinesin family member C1 | NM_002263 | Hs00382565_m1 |
| KIF2C | kinesin family member 2C | NM_006845 | Hs00199232_m1 |
| KIFC14 | kinesin family member 14 | NM_014875 | Hs00208408_m1 |
| CCNB2 | cyclin B2 | NM_004701 | Hs00270424_m1 |
| SIL | TAL1 (SCL) interrupting locus | NM_003035 | Hs00161700_m1 |
| TNPO1 | transportin 1 | NM_002270 | Hs00266970_m1 |
| LMNB1 | lamin B1 | NM_005573 | Hs00194369_m1 |
| CDH2 | cadherin 2, type 1, | NM_001792 | Hs00169953_m1 |
| | N-cadherin (neuronal) | | |
| FALZ | fetal Alzheimer antigen | NM_004459 | Hs00189461_m1 |
| SGNE1 | secretory granule, neuroendocrine | NM_003020 | Hs00161638_m1 |
| | protein 1 (7B2 protein) | | |
| ADAM8 | a disintegrin and | NM_001109 | Hs00174246_m1 |
| | metalloproteinase domain 8 | | |
| SPP1 | secreted phosphoprotein 1 | NM_001040058, | Hs00167093_m1 |
| | (osteopontin, bone sialoprotein I, | NM_000582 | |
| | early T-lymphocyte activation 1) | | |
| ACTB | actin, beta | NM_001101 | Hs99999903_m1 |
| HPRT1 | hypoxanthine | NM_000194 | Hs99999909_m1 |
| | phosphoribosyltransferase 1 | | |
| | (Lesch-Nyhan syndrome) | | |

For each gene, the RefSeq accession for the mRNA sequence was used as the basis for the design of the primer and probe sequences. All of the primer and probe sets listed above were designed and manufactured by Applied Biosystems as part of the TaqMan Gene Expression Assays. These assays can be found at https://products.appliedbiosystems.com.

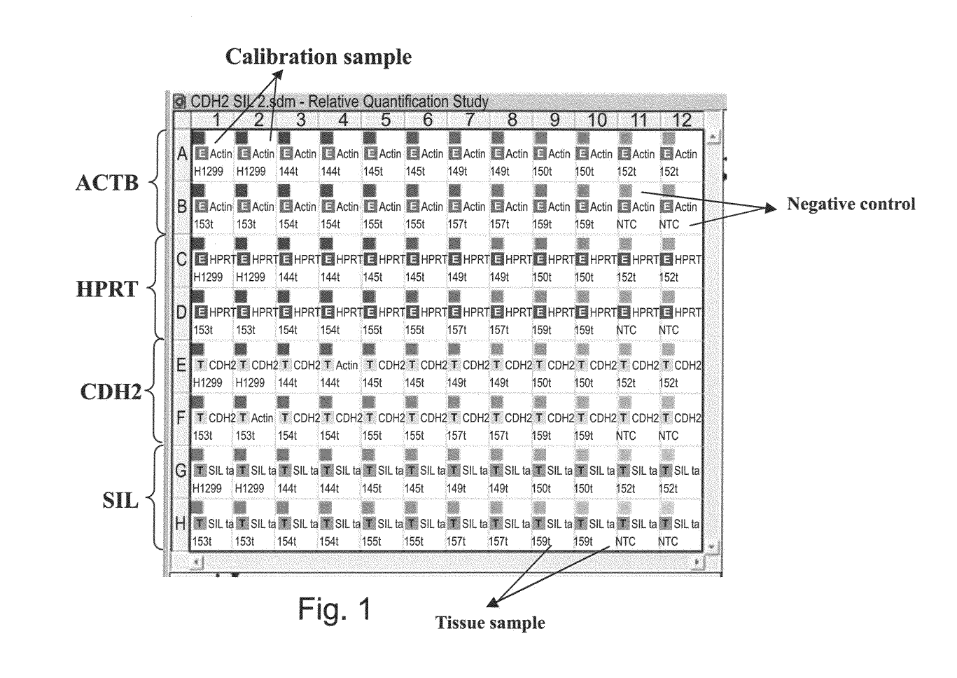

Experiments were performed on 96-well plates containing duplicates for 10 tumor samples, a sample of H1299, and a negative control (no cDNA). In each plate two target genes and two endogenous control genes ActB and HPRT1 were tested (FIG. 1). To control for possible variations among PCR runs in different plates, the expression of all the analyzed and endogenous control genes was compared to their expression in the H1299 cell line included in every plate. Since the normalization to the endogenous control genes ACTB and HPRT1 led to similar results and conclusions, the presented data was normalized to the mean of both of these genes. The relative gene expression for all analyzed genes was calculated using the “Relative Quantification Study” program (SDS 2.2 software applications).

Statistical Analysis:

The expression data for most of the genes (CDH2, SIL, TNPO1, SPP1, KIFC1, KIF2C, KIF14, LMNB, CCNB2, SGNE1) was normalized using log 10 transforms. The FALZ and ADAM8 expression data was normalized using a square root transformation (Table 3, herein below; FIG. 2).

TABLE 3

| | | | | VALUES AFTER | |
|---|---|---|---|---|---|
| | | ABSOLUTE VALUES | | NORMALIZATION | |
| GENE | N | MEAN | SD | MEAN | SD |
| KIFC1 | 140 | 0.437 | 0.509 | −0.520* | 0.352 |
| KIF2C | 142 | 0.160 | 0.208 | −0.981* | 0.390 |
| KIF14 | 140 | 0.470 | 0.603 | −0.598* | 0.533 |
| CCNB2 | 142 | 0.393 | 0.358 | −0.545* | 0.351 |
| SIL | 142 | 0.390 | 0.357 | −0.54** | 0.327 |
| TNPO1 | 142 | 3.060 | 3.412 | 0.359* | 0.298 |
| LMNB1 | 142 | 0.676 | 0.452 | −0.239* | 0.233 |
| CDH2 | 142 | 0.854 | 2.295 | −0.516* | 0.540 |
| FALZ | 142 | 2.422 | 2.366 | 1.44** | 0.590 |
| SGNE1 | 142 | 7.999 | 13.720 | 0.627* | 0.460 |
| ADAM8 | 141 | 82.194 | 71.86 | 8.318** | 3.618 |
| SPP1 | 142 | 414.177 | 528.82 | 2.311* | 0.553 |

*Calculated using log (base 10) of absolute value
**Calculated using square root of absolute value

Univariate Cox regression analyses were run for each gene and summary expression variable, with time to diagnosis of brain metastasis as the dependent variable. Genes and expression variables achieving p-values of 0.1 or lower were then entered as potential predictors in a stepwise, multivariate Cox regression analysis, which retained those predictors that had p-values <0.05. The resulting regression equation was used to define a predictive score. Life table analysis and Kaplan-Meier plots were used to compare the subjects with high, intermediate and low scores. Further analyses considered the predictive ability of the score separately in patients with Stage I/II and Stage III/IV cancer. Life table analysis and Kaplan-Meier plots were used to compare subjects with high and low expression levels of CDH2 protein. The statistical analysis was performed using SPSS (version 14.0) software.

Results

Gene Expression Studies on Affymetrix U95 Chips Table 4 herein below lists all the genes that were up-regulated by at least 2 fold in primary lung tumor samples with brain metastasis as compared to primary lung tumor samples without brain metastasis.

TABLE 4

| REFSEQ | GENENAME |
|---|---|
| NM_000582.2 | secreted phosphoprotein 1 (osteopontin, bone |
| | sialoprotein I, early T-lymphocyte activation 1) |
| NM_000582.2 | secreted phosphoprotein 1 (osteopontin, bone |
| | sialoprotein I, early T-lymphocyte activation 1) |
| NM_003325.3 | HIR histone cell cycle regulation defective homolog A |
| | (S. cerevisiae) |
| NM_001618.2 | ADP-ribosyltransferase (NAD+; poly (ADP-ribose) |
| | polymerase) |
| NM_001949.2 | E2F transcription factor 3 |
| NM_001618.2 | ADP-ribosyltransferase (NAD+; poly (ADP-ribose) |
| | polymerase) |
| NM_013285.1 | nucleolar GTPase |
| NM_014288.3 | integrin beta 3 binding protein (beta3-endonexin) |
| NM_001792.2 | cadherin 2, type 1, N-cadherin (neuronal) |
| NM_001394.4, NM_001394.4 | dual specificity phosphatase 4 |
| NM_000143.2 | fumarate hydratase |
| NM_022782.2 | M-phase phosphoprotein 9 |
| NM_005950.1, NM_005950.1 | metallothionein 1G |
| NM_005956.2 | methylenetetrahydrofolate dehydrogenase (NADP+ |
| | dependent), methenyltetrahydrofolate cyclohydrolase, |
| | formyltetrahydrofolate synthetase |
| NM_003020.1 | secretory granule, neuroendocrine protein 1 (7B2 |
| | protein) |
| NM_002370.2 | mago-nashi homolog, proliferation-associated |
| | (Drosophila) |
| NM_000436.1 | 3-oxoacid CoA transferase |
| NM_004446.1 | glutamyl-prolyl-tRNA synthetase |
| NM_014849.2 | synaptic vesicle glycoprotein 2A |
| NM_006198.1 | Purkinje cell protein 4 |
| NM_004687.3 | myotubularin related protein 4 |
| NM_025073.1 | hypothetical protein FLJ21168 |
| NM_152902.2 | putative MAPK activating protein |
| NM_000987.2 | ribosomal protein L26 |
| NM_006402.2 | hepatitis B virus x interacting protein |
| NM_014255.3 | transmembrane protein 4 |
| NM_006793.2, NM_006793.2 | peroxiredoxin 3 |
| NM_024923.2 | nucleoporin 210 |
| NM_006335.1 | translocase of inner mitochondrial membrane 17 |
| | homolog A (yeast) |
| NM_006701.2, NM_006701.2, | thioredoxin-like 4 |
| XM_351169 | |
| NM_003094.1 | small nuclear ribonucleoprotein polypeptide E |
| NM_006519.1 | t-complex-associated-testis-expressed 1-like 1 |
| NM_018230.2 | nucleoporin 133 kDa |
| NM_007100.1 | ATP synthase, H+ transporting, mitochondrial F0 |
| | complex, subunit e |
| NM_001606.2 | ATP-binding cassette, sub-family A (ABC1), member 2 |
| NM_015475.1, NM_015475.1 | DKFZP564F0522 protein |
| NM_006118.2 | HS1 binding protein |
| NM_014455.1 | zinc finger protein 364 |
| NM_017572.2, NM_017572.2 | MAP kinase-interacting serine/threonine kinase 2 |
| NM_006014.2 | DNA segment on chromosome X (unique) 9879 |
| | expressed sequence |
| NM_014707.1, NM_014707.1, | histone deacetylase 9 |
| NM_058176.1, NM_058177.1, | |
| NM_178423.1 | |
| | hypothetical protein LOC155060 |
| NM_012413.2 | glutaminyl-peptide cyclotransferase (glutaminyl |
| | cyclase) |
| NM_003797.2, NM_003797.2 | embryonic ectoderm development |
| NM_016936.2 | ubinuclein 1 |
| NM_015014.1, NM_015014.1 | KIAA0117 protein |
| NM_014937.2, NM_014937.2, | inositol polyphosphate-5-phosphatase F |
| NM_198330.1 | |
| NM_000443.2, NM_000443.2, | ATP-binding cassette, sub-family B (MDR/TAP), |
| NM_018849.1 | member 4 |
| NM_005673, NM_005673 | solute carrier family 25 (mitochondrial carrier; Graves |
| | disease autoantigen), member 16 |
| NM_175898.2 | hypothetical protein LOC283687 |
| NM_000195.2, NM_000195.2, | Hermansky-Pudlak syndrome 1 |
| NM_182637.1, NM_182638.1 | |
| XM_055195.6 | exportin 6 |
| NM_003073.2 | SWI/SNF related, matrix associated, actin dependent |
| | regulator of chromatin, subfamily b, member 1 |
| NM_033512.1 | KIAA1750 protein |
| NM_002819.3, NM_002819.3, | polypyrimidine tract binding protein 1 |
| NM_031990.2, NM_031991.2 | |
| NM_032778.3, NM_032778.3 | myc-induced nuclear antigen, 53 kDa |
| NM_014661.2 | KIAA0140 gene product |
| XM_059095.8 | formin binding protein 2 |
| NM_000233.1 | luteinizing hormone/choriogonadotropin receptor |
| NM_006012.1 | ClpP caseinolytic protease, ATP-dependent, proteolytic |
| | subunit homolog (E. coli) |
| NM_002791.1 | proteasome (prosome, macropain) subunit, alpha type, 6 |
| NM_032815.2 | hypothetical protein FLJ14639 |
| NM_000314.2 | phosphatase and tensin homolog (mutated in multiple |
| | advanced cancers 1) |
| NM_030809.1 | chromosome 12 open reading frame 22 |
| | USP6 N-terminal like |
| NM_003434.3 | zinc finger protein 133 (clone pHZ-13) |
| NM_006414.1, NM_006414.1 | ribonuclease P (38 kD) |
| NM_014283.2, NM_014283.2 | chromosome 1 open reading frame 9 |
| NM_014351.2, NM_014351.2 | sulfotransferase family 4A, member 1 |
| NM_001792.2 | cadherin 2, type 1, N-cadherin (neuronal) |
| NM_007100.1 | ATP synthase, H+ transporting, mitochondrial F0 |
| | complex, subunit e |
| NM_005784, NM_005784 | mutL homolog 3 (E. coli) |
| NM_005649.2 | zinc finger protein 354A |
| NM_000505.2 | coagulation factor XII (Hageman factor) |
| NM_014409.2 | TAF5-like RNA polymerase II, p300/CBP-associated |
| | factor (PCAF)-associated factor, 65 kDa |
| NM_000447.1, NM_000447.1 | presenilin 2 (Alzheimer disease 4) |
| NM_019116.1 | similar to ubiquitin binding protein |
| NM_006441.1 | 5,10-methenyltetrahydrofolate synthetase (5- |
| | formyltetrahydrofolate cyclo-ligase) |
| NM_174931.1 | hypothetical protein FLJ38348 |
| NM_006701.2, NM_006701.2, | thioredoxin-like 4 |
| XM_351169 | |
| NM_001329.1, NM_001329.1 | C-terminal binding protein 2 |
| | KIAA1041 protein |
| NM_012248.2 | selenophosphate synthetase 2 |
| NM_007049.2, NM_007049.2 | butyrophilin, subfamily 2, member A1 |
| NM_000828.2, NM_000828.2, | glutamate receptor, ionotrophic, AMPA 3 |
| NM_007325.2 | |
| | zinc finger, BED domain containing 4 |
| NM_015636.2, NM_015636.2 | eukaryotic translation initiation factor 2B, subunit 4 |
| | delta, 67 kDa |
| NM_003423.1 | zinc finger protein 43 (HTF6) |
| NM_006383.1 | DNA-dependent protein kinase catalytic subunit- |
| | interacting protein 2 |
| NM_021111.1 | reversion-inducing-cysteine-rich protein with kazal |
| | motifs |
| NM_004094.3 | eukaryotic translation initiation factor 2, subunit 1 |
| | alpha, 35 kDa |
| NM_012311.2 | KIN, antigenic determinant of recA protein homolog |
| | (mouse) |
| NM_006808.2 | Sec61 beta subunit |
| NM_004703.2 | rabaptin, RAB GTPase binding effector protein 1 |
| NM_015093.2, NM_015093.2 | mitogen-activated protein kinase kinase kinase 7 |
| | interacting protein 2 |
| NM_007359.3 | cancer susceptibility candidate 3 |
| NM_001655.3 | archain 1 |
| NM_015542.2, NM_015542.2 | UPF2 regulator of nonsense transcripts homolog |
| | (yeast) |
| NM_002954.3 | ribosomal protein S27a |
| NM_006246.2 | protein phosphatase 2, regulatory subunit B (B56), |
| | epsilon isoform |
| NM_006911.2 | relaxin 1 (H1) |
| NM_007080.1 | LSM6 homolog, U6 small nuclear RNA associated (S. cerevisiae) |
| NM_014737.1, NM_014737.1, | Ras association (RalGDS/AF-6) domain family 2 |
| NM_170773.1 | |
| NM_001638.1 | apolipoprotein F |
| NM_021074.1 | NADH dehydrogenase (ubiquinone) flavoprotein 2, |
| | 24 kDa |
| NM_005004.1 | NADH dehydrogenase (ubiquinone) 1 beta |
| | subcomplex, 8, 19 kDa |
| NM_001572.2, NM_001572.2, | interferon regulatory factor 7 |
| NM_004029.1, NM_004030.1 | |
| NM_021148.1 | zinc finger protein 273 |
| NM_024079.2 | asparagine-linked glycosylation 8 homolog (yeast, |
| | alpha-1,3-glucosyltransferase) |
| NM_005857.2 | zinc metalloproteinase (STE24 homolog, yeast) |
| NM_001987.3 | ets variant gene 6 (TEL oncogene) |
| | zinc finger protein 450 |
| NM_003276.1 | thymopoietin |
| NM_019107.1, NM_019107.1 | chromosome 19 open reading frame 10 |
| XM_037759.5 | KIAA0376 protein |
| NM_021164.2, NM_021164.2, | splicing factor 4 |
| NM_172231.2 | |
| NM_005920.2 | MADS box transcription enhancer factor 2, |
| | polypeptide D (myocyte enhancer factor 2D) |
| NM_015077.1 | sterile alpha and TIR motif containing 1 |
| NM_144573.1 | nexilin (F actin binding protein) |
| NM_004477.1 | FSHD region gene 1 |
| NM_005389.1 | protein-L-isoaspartate (D-aspartate) O- |
| | methyltransferase |
| | KIAA0543 protein |
| NM_002546.2 | tumor necrosis factor receptor superfamily, member |
| | 11b (osteoprotegerin) |
| XM_170681.5 | DKFZP586P0123 protein |
| NM_001968.1 | eukaryotic translation initiation factor 4E |
| NM_019005.1 | hypothetical protein FLJ20323 |
| NM_005491.1 | chromosome X open reading frame 6 |
| NM_006251.2 | protein kinase, AMP-activated, alpha 1 catalytic |
| | subunit |
| NM_002463.1 | myxovirus (influenza virus) resistance 2 (mouse) |
| NM_021003.2, NM_021003.2, | protein phosphatase 1A (formerly 2C), magnesium- |
| NM_177951.1 | dependent, alpha isoform |
| NM_007346.2 | opioid growth factor receptor |
| NM_006608.1 | putative homeodomain transcription factor 1 |
| NG_000858.1 | dihydrofolate reductase pseudogene 1 |
| NM_003288.2, NM_003288.2, | tumor protein D52-like 2 |
| NM_199359.1, NM_199360.1, | |
| NM_199361.1, NM_199362.1 | |
| NM_004759.3, NM_004759.3 | mitogen-activated protein kinase-activated protein |
| | kinase 2 |
| | HIV-1 induced protein HIN-1 |
| NM_015484.1 | GCIP-interacting protein p29 |
| NM_001516.3 | general transcription factor IIH, polypeptide 3, 34 kDa |
| NM_006978.1 | zinc finger protein 183 (RING finger, C3HC4 type) |
| NM_004287.2, NM_004287.2 | golgi SNAP receptor complex member 2 |
| NM_004124.2 | glia maturation factor, beta |
| NM_002396.2 | malic enzyme 2, NAD(+)-dependent, mitochondrial |
| NM_002934.1 | ribonuclease, RNase A family, 2 (liver, eosinophil- |
| | derived neurotoxin) |
| NM_004580.3, NM_004580.3, | RAB27A, member RAS oncogene family |
| NM_183234.1, NM_183235.1 | |
| NM_000785.2 | cytochrome P450, family 27, subfamily B, polypeptide 1 |
| NM_000449.2 | regulatory factor X, 5 (influences HLA class II |
| | expression) |
| NM_014077.1 | family with sequence similarity 32, member A |
| NM_002572.1 | platelet-activating factor acetylhydrolase, isoform Ib, |
| | beta subunit 30 kDa |
| NM_002377.2 | MAS1 oncogene |
| NM_001566.1, NM_001566.1 | inositol polyphosphate-4-phosphatase, type I, 107 kDa |
| NM_004820.2 | cytochrome P450, family 7, subfamily B, polypeptide 1 |
| NM_012231.2, NM_012231.2 | PR domain containing 2, with ZNF domain |
| NM_005089.1 | U2(RNU2) small nuclear RNA auxiliary factor 1-like 2 |
| NM_006221.1 | protein (peptidyl-prolyl cis/trans isomerase) NIMA- |
| | interacting 1 |
| NM_003821.4 | receptor-interacting serine-threonine kinase 2 |
| NM_000027.2 | aspartylglucosaminidase |
| XM_059929.5 | hypothetical protein LOC137886 |
| NM_002249.3, NM_002249.3 | potassium intermediate/small conductance calcium- |
| | activated channel, subfamily N, member 3 |
| NM_002219, NM_002219 | integral membrane protein 1 |
| | KIAA0644 gene product |
| NM_002818.2 | proteasome (prosome, macropain) activator subunit 2 |
| | (PA28 beta) |
| NM_004232.2 | suppressor of cytokine signaling 4 |
| NM_012160.3 | F-box and leucine-rich repeat protein 4 |
| NM_000327.2 | retinal outer segment membrane protein 1 |
| NM_007131.2 | zinc finger protein 75 (D8C6) |
| NM_002059.3, NM_002059.3, | growth hormone 2 |
| NM_022556.2, NM_022557.2 | |
| NM_021003.2, NM_021003.2, | protein phosphatase 1A (formerly 2C), magnesium- |
| NM_177951.1 | dependent, alpha isoform |
| NM_004619.2, NM_004619.2 | TNF receptor-associated factor 5 |
| NM_003252.2, NM_003252.2 | TIA1 cytotoxic granule-associated RNA binding |
| | protein-like 1 |
| NM_000260.1 | myosin VIIA (Usher syndrome 1B (autosomal |
| | recessive, severe)) |
| NM_004914.2 | RAB36, member RAS oncogene family |
| NM_003571.2 | beaded filament structural protein 2, phakinin |
| NM_015864.2, NM_015864.2 | chromosome 6 open reading frame 32 |
| NM_006785.2, NM_006785.2 | mucosa associated lymphoid tissue lymphoma |
| | translocation gene 1 |
| NM_003762.2, NM_003762.2 | vesicle-associated membrane protein 4 |
| XM_290800.3 | hypothetical protein LOC339287 |
| NM_004365.1 | centrin, EF-hand protein, 3 (CDC31 homolog, yeast) |
| NM_002460.1 | interferon regulatory factor 4 |
| NM_018355.2 | zinc finger protein 415 |
| NM_001976.2, NM_001976.2 | enolase 3, (beta, muscle) |
| NM_003846.1 | peroxisomal biogenesis factor 11B |
| NM_002201.4 | interferon stimulated gene 20 kDa |
| NM_001666.2 | Rho GTPase activating protein 4 |
| NM_032804.4 | chromosome 10 open reading frame 22 |
| NM_014781.3 | RB1-inducible coiled-coil 1 |
| NM_015485.2 | RWD domain containing 3 |
| NM_002806.2 | proteasome (prosome, macropain) 26S subunit, |
| | ATPase, 6 |
| NM_020432.2 | putative homeodomain transcription factor 2 |
| | hypothetical protein LOC283824 |
| NM_018982.3 | hypothetical protein DJ167A19.1 |
| NM_003559.3, NM_003559.3 | phosphatidylinositol-4-phosphate 5-kinase, type II, |
| | beta |
| NM_006785.2, NM_006785.2 | mucosa associated lymphoid tissue lymphoma |
| | translocation gene 1 |
| NM_025032.1 | hypothetical protein FLJ21272 |
| NM_003401.2, NM_003401.2, | X-ray repair complementing defective repair in |
| NM_022406.1 | Chinese hamster cells 4 |
| NM_001109.1 | a disintegrin and metalloproteinase domain 8 |
| NM_012398.1 | phosphatidylinositol-4-phosphate 5-kinase, type I, |
| | gamma |
| NM_007237.2 | SP140 nuclear body protein |
| NM_005931.2 | MHC class I polypeptide-related sequence B |
| NM_016133.2 | insulin induced gene 2 |
| NM_001991.2 | enhancer of zeste homolog 1 (Drosophila) |
| NM_004890.1 | sperm associated antigen 7 |
| NM_004509.2, NM_004509.2, | SP110 nuclear body protein |
| NM_004510.2 | |
| NM_007373.2 | soc-2 suppressor of clear homolog (C. elegans) |
| NM_014716.2 | centaurin, beta 1 |
| NM_021727.3 | fatty acid desaturase 3 |
| NM_014885.1 | anaphase-promoting complex subunit 10 |
| NM_000274.1 | ornithine aminotransferase (gyrate atrophy) |
| NM_002484.1 | nucleotide binding protein 1 (MinD homolog, E. coli) |
| NM_003759.1 | solute carrier family 4, sodium bicarbonate |
| | cotransporter, member 4 |
| NM_018364.2 | hypothetical protein FLJ11220 |
| NM_003341.3, NM_003341.3 | ubiquitin-conjugating enzyme E2E 1 (UBC4/5 |
| | homolog, yeast) |
| NM_002211.2, NM_002211.2, | integrin, beta 1 (fibronectin receptor, beta polypeptide, |
| NM_033666.1, NM_033667.1, | antigen CD29 includes MDF2, MSK12) |
| NM_033668.1, NM_033669.1 | |
| NM_007315.2, NM_007315.2 | signal transducer and activator of transcription 1, |
| | 91 kDa |
| NM_014412.1 | Siah-interacting protein |
| NM_004154.3, NM_004154.3, | pyrimidinergic receptor P2Y, G-protein coupled, 6 |
| NM_176796.1, NM_176797.1 | |
| NM_004310.1 | ras homolog gene family, member H |
| NM_000161.1 | GTP cyclohydrolase 1 (dopa-responsive dystonia) |
| NM_021194.1 | solute carrier family 30 (zinc transporter), member 1 |
| NM_014243.1 | a disintegrin-like and metalloprotease (reprolysin type) |
| | with thrombospondin type 1 motif, 3 |
| NM_015099.2 | calmodulin binding transcription activator 2 |
| NM_021132.1 | protein phosphatase 3 (formerly 2B), catalytic subunit, |
| | beta isoform (calcineurin A beta) |
| NM_015285.1, NM_015285.1 | WD repeat domain 7 |
| NM_000662.4 | N-acetyltransferase 1 (arylamine N-acetyltransferase) |
| NM_030797.1 | hypothetical protein DKFZp566A1524 |
| NM_004334.1 | bone marrow stromal cell antigen 1 |
| NM_014011.4, NM_014011.4 | suppressor of cytokine signaling 5 |
| NM_012425.3, NM_012425.3 | Ras suppressor protein 1 |
| NM_003741.2, NM_003741.2, | chordin |
| NM_177978.1 | |
| NM_002925.2 | regulator of G-protein signalling 10 |
| NM_004316.1 | achaete-scute complex-like 1 (Drosophila) |
| NM_015235.1 | cleavage stimulation factor, 3′ pre-RNA, subunit 2, |
| | 64 kDa, tau variant |
| NM_005013.1 | nucleobindin 2 |
| XM_094581.4 | SEC24 related gene family, member A (S. cerevisiae) |
| NM_005159.2 | actin, alpha, cardiac muscle |
| NM_001560.2 | interleukin 13 receptor, alpha 1 |
| NM_003744.3 | numb homolog (Drosophila) |
| NM_006471.2 | myosin regulatory light chain MRCL3 |
| NM_002303.2 | leptin receptor |
| XM_290895.2 | phosphodiesterase 4D interacting protein |
| | (myomegalin) |
| NM_012420.1 | interferon-induced protein with tetratricopeptide |
| | repeats 5 |
| NM_020980.2 | aquaporin 9 |
| NM_003014.2 | secreted frizzled-related protein 4 |
| NM_001881.2, NM_001881.2, | cAMP responsive element modulator |
| NM_182720.1, NM_182722.1, | |
| NM_182724.1, NM_183060.1, | |
| NM_183013.1, NM_183012.1, | |
| NM_183011.1, NM_182853.1, | |
| NM_182850.1, NM_182772.1, | |
| NM_182771.1, NM_182770.1, | |
| NM_182769.1, NM_182725.1, | |
| NM_182723.1, NM_182721.1, | |
| NM_182719.1, NM_181571.1, | |
| NM_182717.1 | |
| NM_004120.3 | guanylate binding protein 2, interferon-inducible |
| NM_001156.2, NM_001156.2 | annexin A7 |
| NM_000206.1 | interleukin 2 receptor, gamma (severe combined |
| | immunodeficiency) |
| NM_000305.1 | paraoxonase 2 |
| NM_000300.2 | phospholipase A2, group IIA (platelets, synovial fluid) |
| | SH3 multiple domains 3 |
| NM_000873.2 | intercellular adhesion molecule 2 |
| NM_001854.2, NM_001854.2, | collagen, type XI, alpha 1 |
| NM_080629.1 | |
| NM_001710.3 | B-factor, properdin |
| NM_006379.2 | sema domain, immunoglobulin domain (Ig), short basic |
| | domain, secreted, (semaphorin) 3C |

Table 5 herein below lists all the genes that were down-regulated by at least 2 fold in primary lung tumor samples with brain metastasis as compared to primary lung tumor samples without brain metastasis.

TABLE 5

| REFSEQ | GENENAME |
|---|---|
| NM_003252.2, NM_003252.2 | TIA1 cytotoxic granule-associated RNA |
| | binding protein-like 1 |
| NM_002760.2 | protein kinase, Y-linked |
| NM_000782.2 | cytochrome P450, family 24, subfamily A, |
| | polypeptide 1 |
| NM_014608.1 | cytoplasmic FMR1 interacting protein 1 |
| NM_012253.1 | transketolase-like 1 |
| NM_003670.1 | basic helix-loop-helix domain containing, class |
| | B, 2 |
| NM_002169.1 | interferon, alpha 5 |
| | immunoglobulin heavy constant gamma 3 |
| | (G3m marker) |
| XM_374409.1, XM_374409.1, | phosphoribosyl pyrophosphate synthetase 1- |
| NM_175886.2 | like 1 |
| NM_001131.2, NM_001131.2 | cysteine-rich secretory protein 1 |
| NM_003507.1 | frizzled homolog 7 (Drosophila) |
| NM_007031.1 | heat shock transcription factor 2 binding |
| | protein |
| NM_007153.1 | zinc finger protein 208 |
| NM_014648.2 | zinc finger DAZ interacting protein 3 |
| NM_005142.2 | gastric intrinsic factor (vitamin B synthesis) |
| NM_153263.1 | zinc finger protein 549 |
| NM_002518.2, NM_002518.2 | neuronal PAS domain protein 2 |
| NM_015931.1 | fls485 |
| NM_002977.1 | sodium channel, voltage-gated, type IX, alpha |
| NM_005230.1 | ELK3, ETS-domain protein (SRF accessory |
| | protein 2) |
| NM_005275.2 | guanine nucleotide binding protein-like 1 |
| NM_004311.2 | ADP-ribosylation factor-like 3 |
| NM_007360.1 | killer cell lectin-like receptor subfamily K, |
| | member 1 |
| NM_024501.1 | homeo box D1 |
| NM_003057.2, NM_003057.2 | solute carrier family 22 (organic cation |
| | transporter), member 1 |
| NM_005270.2, NM_005270.2, | GLI-Kruppel family member GLI2 |
| NM_030379.1, NM_030380.1 | |
| NM_005038.1 | peptidylprolyl isomerase D (cyclophilin D) |
| NM_006080.1 | sema domain, immunoglobulin domain (Ig), |
| | short basic domain, secreted, (semaphorin) 3A |
| NM_198215.1, NM_198215.1 | family with sequence similarity 13, member |
| | C1 |
| NM_001402.4 | eukaryotic translation elongation factor 1 alpha 1 |
| NM_000212.1 | integrin, beta 3 (platelet glycoprotein IIIa, |
| | antigen CD61) |
| NM_006278.1 | sialyltransferase 4C (beta-galactoside alpha- |
| | 2,3-sialyltransferase) |
| NM_004944.1 | deoxyribonuclease I-like 3 |
| NM_024094.1 | defective in sister chromatid cohesion homolog |
| | 1 (S. cerevisiae) |
| | kinesin family member 1C |
| NM_006516.1 | solute carrier family 2 (facilitated glucose |
| | transporter), member 1 |
| NM_000107.1 | damage-specific DNA binding protein 2, |
| | 48 kDa |
| NM_016341.2 | phospholipase C, epsilon 1 |
| NM_000818.1 | glutamate decarboxylase 2 (pancreatic islets |
| | and brain, 65 kDa) |
| NM_005328.1 | hyaluronan synthase 2 |
| NM_002210.2 | integrin, alpha V (vitronectin receptor, alpha |
| | polypeptide, antigen CD51) |
| NM_000957.2, NM_000957.2, | prostaglandin E receptor 3 (subtype EP3) |
| NM_198712.1, NM_198713.1, | |
| NM_198714.1, NM_198715.1, | |
| NM_198716.1, NM_198717.1, | |
| NM_198718.1, NM_198719.1 | |
| NM_024060.1 | hypothetical protein MGC5395 |
| NM_000537.2 | renin |
| XM_370659.1, XM_370659.1, | KIAA0999 protein |
| NM_025164.3 | |
| NM_014551.3, NM_014551.3 | hypothetical protein 384D8_6 |
| NM_015478.4, NM_015478.4 | l(3)mbt-like (Drosophila) |
| NM_000462.2, NM_000462.2, | ubiquitin protein ligase E3A (human papilloma |
| NM_130838.1 | virus E6-associated protein, Angelman |
| | syndrome) |
| NM_002559.2 | purinergic receptor P2X, ligand-gated ion |
| | channel, 3 |
| NM_004737.2, NM_004737.2 | like-glycosyltransferase |
| NM_004656.2 | BRCA1 associated protein-1 (ubiquitin |
| | carboxy-terminal hydrolase) |
| NM_003786.2, NM_003786.2, | ATP-binding cassette, sub-family C |
| NM_020037.1 | (CFTR/MRP), member 3 |
| NM_014292.2 | chromobox homolog 6 |
| NM_005063.3 | stearoyl-CoA desaturase (delta-9-desaturase) |
| NM_004617.2 | transmembrane 4 superfamily member 4 |
| NM_002405.2 | manic fringe homolog (Drosophila) |
| NM_019094.3, NM_019094.3 | nudix (nucleoside diphosphate linked moiety |
| | X)-type motif 4 |
| NM_032814.1 | hypothetical protein FLJ14627 |
| NM_005797.2, NM_005797.2 | epithelial V-like antigen 1 |
| NM_000312.1 | protein C (inactivator of coagulation factors Va |
| | and VIIIa) |
| NM_019848.2 | solute carrier family 10 (sodium/bile acid |
| | cotransporter family), member 3 |
| NM_012448.3 | signal transducer and activator of transcription |
| | 5B |
| NM_007032.3, NM_007032.3 | Tara-like protein |
| NM_012448.3 | signal transducer and activator of transcription |
| | 5B |
| NM_000684.1 | adrenergic, beta-1-, receptor |
| NM_002976.1 | sodium channel, voltage-gated, type VII, alpha |
| NM_000214.1 | jagged 1 (Alagille syndrome) |
| NM_003239.1 | transforming growth factor, beta 3 |
| NM_001395.1 | dual specificity phosphatase 9 |
| NM_000141.2, NM_000141.2, | fibroblast growth factor receptor 2 (bacteria- |
| NM_023031.1, NM_023030.1, | expressed kinase, keratinocyte growth factor |
| NM_023029.1, NM_023028.1, | receptor, craniofacial dysostosis 1, Crouzon |
| NM_022976.1, NM_022975.2, | syndrome, Pfeiffer syndrome, Jackson-Weiss |
| NM_022970.1, NM_022971.1, | syndrome) |
| NM_022972.1, NM_022974.1, | |
| NM_022973.1 | |
| NM_006387.4 | calcium homeostasis endoplasmic reticulum |
| | protein |
| NM_000461.2 | thyroid hormone receptor, beta (erythroblastic |
| | leukemia viral (v-erb-a) oncogene homolog 2, |
| | avian) |
| NM_004434.1 | echinoderm microtubule associated protein like 1 |
| NM_014459.2 | protocadherin 17 |
| NM_000890.3 | potassium inwardly-rectifying channel, |
| | subfamily J, member 5 |
| NM_000142.2, NM_000142.2 | fibroblast growth factor receptor 3 |
| | (achondroplasia, thanatophoric dwarfism) |
| NM_014738.2 | KIAA0195 gene product |
| NM_007170.1 | testis-specific kinase 2 |
| NM_019025.2, NM_019025.2, | spermine oxidase |
| NM_175839.1, NM_175840.1, | |
| NM_175841.1 | |
| NM_004695.2 | solute carrier family 16 (monocarboxylic acid |
| | transporters), member 5 |
| NM_133455.1 | emilin and multimerin-domain containing |
| | protein 1 |
| NM_183373.2, NM_183373.2 | chromosome 6 open reading frame 145 |
| NM_002594.2 | proprotein convertase subtilisin/kexin type 2 |
| NM_001719.1 | bone morphogenetic protein 7 (osteogenic |
| | protein 1) |
| NM_001674.1, NM_001674.1 | activating transcription factor 3 |
| NM_002757.2, NM_002757.2, | mitogen-activated protein kinase kinase 5 |
| NM_145160.1, NM_145161.1 | |
| NM_002617.3, NM_002617.3 | peroxisome biogenesis factor 10 |
| NM_002405.2 | manic fringe homolog (Drosophila) |
| NM_002757.2, NM_002757.2, | mitogen-activated protein kinase kinase 5 |
| NM_145160.1, NM_145161.1 | |
| NM_003812.1 | a disintegrin and metalloproteinase domain 23 |
| NM_004102.2 | fatty acid binding protein 3, muscle and heart |
| | (mammary-derived growth inhibitor) |
| NM_004431.2 | EphA2 |
| XM_048592.3 | KIAA1045 |
| NM_006225.1 | phospholipase C, delta 1 |
| NM_000599.1 | insulin-like growth factor binding protein 5 |
| NM_005026.2 | phosphoinositide-3-kinase, catalytic, delta |
| | polypeptide |
| NM_000381.1, NM_000381.1, | midline 1 (Opitz/BBB syndrome) |
| NM_033290.1 | |
| NM_004599.2 | sterol regulatory element binding transcription |
| | factor 2 |
| NM_004485.2 | guanine nucleotide binding protein (G protein), |
| | gamma 4 |
| NM_018334.3 | leucine rich repeat neuronal 3 |
| NM_002735.1 | protein kinase, cAMP-dependent, regulatory, |
| | type I, beta |
| NM_003913.3, NM_003913.3 | PRP4 pre-mRNA processing factor 4 homolog |
| | B (yeast) |
| NM_003873.2 | neuropilin 1 |
| NM_175709.1 | chromobox homolog 7 |
| NM_005941.2, NM_005941.2 | matrix metalloproteinase 16 (membrane- |
| | inserted) |
| NM_007065.3 | CDC37 cell division cycle 37 homolog (S. cerevisiae) |
| NM_033100.1 | protocadherin 21 |
| NM_020796.1 | sema domain, transmembrane domain (TM), |
| | and cytoplasmic domain, (semaphorin) 6A |
| NM_002141.2 | homeo box A4 |
| NM_032041.1 | neurocalcin delta |
| NM_006613.2 | GRB2-related adaptor protein |
| NM_000494.2, NM_000494.2 | collagen, type XVII, alpha 1 |
| NM_000141.2, NM_000141.2, | fibroblast growth factor receptor 2 (bacteria- |
| NM_023031.1, NM_023030.1, | expressed kinase, keratinocyte growth factor |
| NM_023029.1, NM_023028.1, | receptor, craniofacial dysostosis 1, Crouzon |
| NM_022976.1, NM_022975.2, | syndrome, Pfeiffer syndrome, Jackson-Weiss |
| NM_022970.1, NM_022971.1, | syndrome) |
| NM_022972.1, NM_022974.1, | |
| NM_022973.1 | |
| NM_003128.1, NM_003128.1 | spectrin, beta, non-erythrocytic 1 |
| NM_000702.1 | ATPase, Na+/K+ transporting, alpha 2 (+) |
| | polypeptide |
| NM_014220.1 | transmembrane 4 superfamily member 1 |
| NM_005902.2 | MAD, mothers against decapentaplegic |
| | homolog 3 (Drosophila) |
| NM_003238.1 | transforming growth factor, beta 2 |
| NM_004429.3 | ephrin-B1 |
| NM_004429.3 | ephrin-B1 |
| NM_002607.2, NM_002607.2 | platelet-derived growth factor alpha |
| | polypeptide |
| NM_012244.2, NM_012244.2 | solute carrier family 7 (cationic amino acid |
| | transporter, y+ system), member 8 |
| NM_000599.1 | insulin-like growth factor binding protein 5 |
| NM_004364.2 | CCAAT/enhancer binding protein (C/EBP), |
| | alpha |
| NM_006749.3 | solute carrier family 20 (phosphate |
| | transporter), member 2 |
| NM_002203.2 | integrin, alpha 2 (CD49B, alpha 2 subunit of |
| | VLA-2 receptor) |
| NM_000142.2, NM_000142.2 | fibroblast growth factor receptor 3 |
| | (achondroplasia, thanatophoric dwarfism) |
| NM_000685.3, NM_000685.3, | angiotensin II receptor, type 1 |
| NM_009585.2, NM_004835.2, | |
| NM_031850.1 | |
| NM_002135.3, NM_002135.3, | nuclear receptor subfamily 4, group A, |
| NM_173157.1 | member 1 |
| NM_012302.1 | latrophilin 2 |
| NM_001730.2 | Kruppel-like factor 5 (intestinal) |
| NM_006186.2, NM_006186.2, | nuclear receptor subfamily 4, group A, |
| NM_173171.1, NM_173172.1 | member 2 |
| NM_003798.1 | catenin (cadherin-associated protein), alpha- |
| | like 1 |
| NM_002135.3, NM_002135.3, | nuclear receptor subfamily 4, group A, |
| NM_173157.1 | member 1 |
| NM_003128.1, NM_003128.1 | spectrin, beta, non-erythrocytic 1 |
| NM_001423.1 | epithelial membrane protein 1 |
| NM_000888.3 | integrin, beta 6 |
| NM_006403.2, NM_006403.2 | neural precursor cell expressed, |
| | developmentally down-regulated 9 |
| NM_006334.2, NM_006334.2, | olfactomedin 1 |
| NM_014279.2 | |
| NM_000213.2 | integrin, beta 4 |
| NM_000141.2, NM_000141.2, | fibroblast growth factor receptor 2 (bacteria- |
| NM_023031.1, NM_023030.1, | expressed kinase, keratinocyte growth factor |
| NM_023029.1, NM_023028.1, | receptor, craniofacial dysostosis 1, Crouzon |
| NM_022976.1, NM_022975.2, | syndrome, Pfeiffer syndrome, Jackson-Weiss |
| NM_022970.1, NM_022971.1, | syndrome) |
| NM_022972.1, NM_022974.1, | |
| NM_022973.1 | |
| NM_002165.2, NM_002165.2 | inhibitor of DNA binding 1, dominant negative |
| | helix-loop-helix protein |
| NM_006732.1 | FBJ murine osteosarcoma viral oncogene |
| | homolog B |
| NM_005031.3, NM_005031.3 | FXYD domain containing ion transport |
| | regulator 1 (phospholemman) |
| NM_001823.3 | creatine kinase, brain |
| NM_000227.2, NM_000227.2 | laminin, alpha 3 |
| NM_001870.1 | carboxypeptidase A3 (mast cell) |
| NM_004093.2 | ephrin-B2 |
| NM_001200.1 | bone morphogenetic protein 2 |
| NM_005252.2 | v-fos FBJ murine osteosarcoma viral oncogene |
| | homolog |
| NM_001233.3, NM_001233.3 | caveolin 2 |
| NM_001200.1 | bone morphogenetic protein 2 |
| NM_006829.1 | adipose specific 2 |
| NM_002165.2, NM_002165.2 | inhibitor of DNA binding 1, dominant negative |
| | helix-loop-helix protein |
| NM_001753.3 | caveolin 1, caveolae protein, 22 kDa |
| NM_006744.2 | retinol binding protein 4, plasma |
| NM_005139.1 | annexin A3 |
| NM_000689.3 | aldehyde dehydrogenase 1 family, member A1 |
| NM_005228.3, NM_005228.3, | epidermal growth factor receptor |
| NM_201283.1, NM_201284.1 | (erythroblastic leukemia viral (v-erb-b) |
| | oncogene homolog, avian) |
| NM_002165.2, NM_002165.2 | inhibitor of DNA binding 1, dominant negative |
| | helix-loop-helix protein |

Table 6 herein below lists all the genes that were up-regulated by at least 2 fold in primary lung tumor samples with brain metastasis as compared to primary lung tumor samples.

TABLE 6

| REFSEQ | GENENAME |
|---|---|
| NM_013285.1 | nucleolar GTPase |
| NM_003325.3 | HIR histone cell cycle regulation |
| | defective homolog A (S. cerevisiae) |
| NM_001949.2 | E2F transcription factor 3 |
| | mediator of DNA damage checkpoint 1 |
| NM_007375.3, NM_007375.3 | TAR DNA binding protein |
| NM_015599.1 | phosphoglucomutase 3 |
| NM_002906.2 | radixin |
| NM_014503.1 | down-regulated in metastasis |
| NM_003437.2 | zinc finger protein 136 (clone pHZ-20) |
| NM_005573.2 | lamin B1 |
| NM_002270.2, NM_002270.2 | transportin 1 |
| NM_030621.2, NM_030621.2 | Dicer1, Dcr-1 homolog (Drosophila) |
| NM_003358.1 | UDP-glucose ceramide |
| | glucosyltransferase |
| NM_004324.3, NM_004324.3, | BCL2-associated X protein |
| NM_138762.2, NM_138764.2, | |
| NM_138765.2, NM_138763.2 | |
| NM_002270.2, NM_002270.2 | transportin 1 |
| NM_006708.1 | glyoxalase I |
| NM_021177.3 | LSM2 homolog, U6 small nuclear |
| | RNA associated (S. cerevisiae) |
| XM_371575.1 | formin binding protein 3 |
| NM_007276.3, NM_007276.3 | chromobox homolog 3 (HP1 gamma |
| | homolog, Drosophila) |
| NM_016076.2 | CGI-146 protein |
| NM_000179.1 | mutS homolog 6 (E. coli) |
| NM_002827.2 | protein tyrosine phosphatase, non- |
| | receptor type 1 |
| NM_014006.2, NM_014006.2 | PI-3-kinase-related kinase SMG-1 |
| NM_002685.1 | polymyositis/scleroderma autoantigen |
| | 2, 100 kDa |
| NM_003583.2, NM_003583.2 | dual-specificity tyrosine-(Y)- |
| | phosphorylation regulated kinase 2 |
| NM_002907.2, NM_002907.2 | RecQ protein-like (DNA helicase Q1- |
| | like) |
| NM_000234.1 | ligase I, DNA, ATP-dependent |
| NM_007358.1 | likely ortholog of mouse metal |
| | response element binding transcription |
| | factor 2 |
| NM_001416.1 | eukaryotic translation initiation factor |
| | 4A, isoform 1 |
| NM_000251.1 | mutS homolog 2, colon cancer, |
| | nonpolyposis type 1 (E. coli) |
| NM_014071.2 | nuclear receptor coactivator 6 |
| NM_006773.3 | DEAD (Asp-Glu-Ala-Asp) box |
| | polypeptide 18 |
| NM_006845.2 | kinesin family member 2C |
| NM_003324.3 | tubby like protein 3 |
| NM_005802.2 | topoisomerase I binding, |
| | arginine/serine-rich |
| NM_006115.2 | preferentially expressed antigen in |
| | melanoma |
| NM_004399.1, NM_004399.1, | DEAD/H (Asp-Glu-Ala-Asp/His) box |
| NM_030655.2 | polypeptide 11 (CHL1-like helicase |
| | homolog, S. cerevisiae) |
| NM_006907.2, NM_006907.2 | pyrroline-5-carboxylate reductase 1 |
| NM_004111.4 | flap structure-specific endonuclease 1 |
| NM_003093.1 | small nuclear ribonucleoprotein |
| | polypeptide C |
| NM_003794.2 | sorting nexin 4 |
| NM_001321.1 | cysteine and glycine-rich protein 2 |
| NM_006256.1 | protein kinase C-like 2 |
| | mediator of DNA damage checkpoint 1 |
| NM_005385.2 | natural killer-tumor recognition |
| | sequence |
| NM_006303.2 | JTV1 gene |
| NM_000520.2 | hexosaminidase A (alpha polypeptide) |
| NM_006625.3, NM_006625.3, | FUS interacting protein (serine- |
| NM_021993 | arginine rich) 1 |
| NM_002945.2 | replication protein A1, 70 kDa |
| NM_003090.1 | small nuclear ribonucleoprotein |
| | polypeptide A′ |
| NM_002754.3 | mitogen-activated protein kinase 13 |
| NM_004459.5, NM_004459.5 | fetal Alzheimer antigen |
| NM_006170.1 | nucleolar protein 1, 120 kDa |
| NM_002865.1 | RAB2, member RAS oncogene family |
| NM_001412.2 | eukaryotic translation initiation factor |
| | 1A |
| NM_000465.1 | BRCA1 associated RING domain 1 |
| NM_004494.1 | hepatoma-derived growth factor (high- |
| | mobility group protein 1-like) |
| NM_001237.2 | cyclin A2 |
| NM_025032.1 | hypothetical protein FLJ21272 |
| NM_004964.2 | histone deacetylase 1 |
| NM_014501.1 | ubiquitin-conjugating enzyme E2S |
| NM_001826.1 | CDC28 protein kinase regulatory |
| | subunit 1B |
| XM_047325.7 | THO complex 2 |
| NM_002916.3, NM_002916.3 | replication factor C (activator 1) 4, |
| | 37 kDa |
| NM_002915.2, NM_002915.2 | replication factor C (activator 1) 3, |
| | 38 kDa |
| NM_004526.2 | MCM2 minichromosome maintenance |
| | deficient 2, mitotin (S. cerevisiae) |
| NM_007111.3 | transcription factor Dp-1 |
| NM_003017.3 | splicing factor, arginine/serine-rich 3 |
| NM_005496.2 | SMC4 structural maintenance of |
| | chromosomes 4-like 1 (yeast) |
| NM_003292.1 | translocated promoter region (to |
| | activated MET oncogene) |
| NM_001569.2 | interleukin-1 receptor-associated |
| | kinase 1 |
| NM_005916.3, NM_005916.3 | MCM7 minichromosome maintenance |
| | deficient 7 (S. cerevisiae) |
| NM_007271.2 | serine/threonine kinase 38 |
| NM_000520.2 | hexosaminidase A (alpha polypeptide) |
| NM_002196.1 | insulinoma-associated 1 |
| NM_004515.2 | interleukin enhancer binding factor 2, |
| | 45 kDa |
| NM_000876.1 | insulin-like growth factor 2 receptor |
| NM_004336.1 | BUB1 budding uninhibited by |
| | benzimidazoles 1 homolog (yeast) |
| NM_003200.1 | transcription factor 3 (E2A |
| | immunoglobulin enhancer binding |
| | factors E12/E47) |
| NM_003610.2 | RAE1 RNA export 1 homolog |
| | (S. pombe) |
| NM_002093.2 | glycogen synthase kinase 3 beta |
| NM_014865.2 | chromosome condensation-related |
| | SMC-associated protein 1 |
| NM_006231.1 | polymerase (DNA directed), epsilon |
| NM_004630.2, NM_004630.2, | splicing factor 1 |
| NM_201995.1, NM_201997.1 | |
| NM_017869.2, NM_017869.2 | BTG3 associated nuclear protein |
| NM_006904.6 | protein kinase, DNA-activated, |
| | catalytic polypeptide |
| NM_004559.2 | nuclease sensitive element binding |
| | protein 1 |
| XM_371575.1 | formin binding protein 3 |
| NM_003431.2 | zinc finger protein 124 (HZF-16) |
| NM_002794.3 | proteasome (prosome, macropain) |
| | subunit, beta type, 2 |
| NM_138779.2 | hypothetical protein BC015148 |
| NM_004053.2 | bystin-like |
| NM_001905.1 | CTP synthase |
| NM_005104.2 | bromodomain containing 2 |
| NM_005104.2 | bromodomain containing 2 |
| NM_005030.3 | polo-like kinase 1 (Drosophila) |
| NM_006444.1 | SMC2 structural maintenance of |
| | chromosomes 2-like 1 (yeast) |
| NM_001269.2 | chromosome condensation 1 |
| NM_006256.1 | protein kinase C-like 2 |
| NM_005850.3 | splicing factor 3b, subunit 4, 49 kDa |
| NM_002497.1 | NIMA (never in mitosis gene a)- |
| | related kinase 2 |
| NM_003362.2, NM_003362.2 | uracil-DNA glycosylase |
| XM_027172.3 | chromosome 1 open reading frame 34 |
| NM_001255.1 | CDC20 cell division cycle 20 homolog |
| | (S. cerevisiae) |
| NM_003324.3 | tubby like protein 3 |
| NM_016343.2 | centromere protein F, 350/400ka |
| | (mitosin) |
| NM_005056.1 | Jumonji, AT rich interactive domain |
| | 1A (RBP2-like) |
| NM_002592.2, NM_002592.2 | proliferating cell nuclear antigen |
| NM_001327.1 | cancer/testis antigen 1 |
| NM_021964.1 | zinc finger protein 148 (pHZ-52) |
| NM_007019.2, NM_007019.2, | ubiquitin-conjugating enzyme E2C |
| NM_181799.1, NM_181800.1, | |
| NM_181801.1, NM_181802.1 | |
| NM_015640.1, NM_015640.1 | PAI-1 mRNA-binding protein |
| NM_002482.2, NM_002482.2, | nuclear autoantigenic sperm protein |
| NM_152298.2 | (histone-binding) |
| NM_004645.1 | coilin |
| XM_373318.1 | KIAA0169 |
| NM_004169.3, NM_004169.3 | serine hydroxymethyltransferase 1 |
| | (soluble) |
| NM_014516.2 | CCR4-NOT transcription complex, |
| | subunit 3 |
| NM_003198.1 | transcription elongation factor B |
| | (SIII), polypeptide 3 (110 kDa, elongin |
| | A) |
| XM_371813.1 | kinesin family member C1 |
| NM_144570.1 | chromosome 16 open reading frame |
| | 34 |
| NM_012112.4 | TPX2, microtubule-associated protein |
| | homolog (Xenopus laevis) |
| NM_004856.4, NM_004856.4 | kinesin family member 23 |
| NM_003158, NM_003158, | serine/threonine kinase 6 |
| NM_003600.2, NM_198433.1, | |
| NM_198434.1, NM_198435.1, | |
| NM_198436.1 | |
| NM_002131.2, NM_002131.2, | high mobility group AT-hook 1 |
| NM_145899.1, NM_145900, | |
| NM_145901.1, NM_145902.1, | |
| NM_145903.1, NM_145904.1 | |
| NM_002093.2 | glycogen synthase kinase 3 beta |
| NM_015013.1 | KIAA0601 protein |
| NM_021953.2, NM_021953.2, | forkhead box M1 |
| NM_202002.1 | |
| NM_002691.1 | polymerase (DNA directed), delta 1, |
| | catalytic subunit 125 kDa |
| NM_005596.1 | nuclear factor I/B |
| NM_002417.2 | antigen identified by monoclonal |
| | antibody Ki-67 |
| NM_001798.2, NM_001798.2 | cyclin-dependent kinase 2 |
| NM_007040.2, NM_007040.2, | E1B-55 kDa-associated protein 5 |
| NM_144732.1, NM_144733.1 | |
| NM_004456.3, NM_004456.3 | enhancer of zeste homolog 2 |
| | (Drosophila) |
| NM_002466.2 | v-myb myeloblastosis viral oncogene |
| | homolog (avian)-like 2 |
| NM_016426.3 | G-2 and S-phase expressed 1 |
| NM_005686.2 | SRY (sex determining region Y)-box |
| | 13 |
| NM_004516.2, NM_004516.2, | interleukin enhancer binding factor 3, |
| NM_012218.2 | 90 kDa |
| NM_003836.3 | delta-like 1 homolog (Drosophila) |
| NM_002388.3 | MCM3 minichromosome maintenance |
| | deficient 3 (S. cerevisiae) |
| NM_015640.1, NM_015640.1 | PAI-1 mRNA-binding protein |
| XM_371813.1 | kinesin family member C1 |
| NM_007370.3, NM_007370.3 | replication factor C (activator 1) 5, |
| | 36.5 kDa |
| NM_002740.3 | protein kinase C, iota |
| NM_002936.3 | ribonuclease H1 |
| | kinesin family member 14 |
| NM_012474.3 | uridine monophosphate kinase |
| NM_000057.1 | Bloom syndrome |
| NM_004217.1 | aurora kinase B |
| NM_006896.2 | homeo box A7 |
| NM_004237.2 | thyroid hormone receptor interactor 13 |
| NM_004341.2 | carbamoyl-phosphate synthetase 2, |
| | aspartate transcarbamylase, and |
| | dihydroorotase |
| NM_004629.1 | Fanconi anemia, complementation |
| | group G |
| NM_000337.3, NM_000337.3 | sarcoglycan, delta (35 kDa dystrophin- |
| | associated glycoprotein) |
| XM_371813.1 | kinesin family member C1 |
| NM_000372.2 | tyrosinase (oculocutaneous albinism |
| | IA) |
| NM_032737.2 | lamin B2 |
| NM_003504.3 | CDC45 cell division cycle |
| | 45-like (S. cerevisiae) |
| NM_003016.1 | splicing factor, arginine/serine-rich 2 |
| NM_005828.1 | WD-repeat protein |
| NM_003579.2 | RAD54-like (S. cerevisiae) |
| NM_001067.2 | topoisomerase (DNA) II alpha 170 kDa |
| NM_004701.2 | cyclin B2 |
| NM_005066.1 | splicing factor proline/glutamine rich |
| | (polypyrimidine tract binding protein |
| | associated) |
| NM_007085.3 | follistatin-like 1 |
| NM_003447.2 | zinc finger protein 165 |
| NM_021244.2 | Ras-related GTP binding D |
| NM_052940.3 | hypothetical protein MGC8974 |
| NM_012291.3 | extra spindle poles like 1 (S. cerevisiae) |
| NM_005884.2 | p21(CDKN1A)-activated kinase 4 |
| NM_014740.2 | DEAD (Asp-Glu-Ala-Asp) box |
| | polypeptide 48 |
| NM_001071.1 | thymidylate synthetase |
| NM_006904.6 | protein kinase, DNA-activated, |
| | catalytic polypeptide |

Table 7 herein below lists all the genes that were down-regulated by at least 2 fold in primary lung tumor samples with brain metastasis as compared to primary lung tumor samples.

TABLE 7

| AFFYID | GENENAME |
|---|---|
| 160029_at | protein kinase C, beta 1 |
| 36896_s_at | aryl hydrocarbon receptor nuclear translocator-like |
| 36142_at | spinocerebellar ataxia 1 (olivopontocerebellar ataxia 1, |
| | autosomal dominant, ataxin 1) |
| 33440_at | transcription factor 8 (represses interleukin 2 expression) |
| 38837_at | hypothetical protein DJ971N18.2 |
| 1252_at | polyposis locus protein 1 |
| 33240_at | likely ortholog of mouse semaF cytoplasmic domain |
| | associated protein 3 |
| 39582_at | cylindromatosis (turban tumor syndrome) |
| 38254_at | KIAA0882 protein |
| 39032_at | transforming growth factor beta-stimulated protein TSC- |
| | 22 |
| 34376_at | protein kinase (cAMP-dependent, catalytic) inhibitor |
| | gamma |
| 39542_at | ectodermal-neural cortex (with BTB-like domain) |
| 33158_at | Kallmann syndrome 1 sequence |
| 36553_at | acetylserotonin O-methyltransferase-like |
| 39634_at | slit homolog 2 (Drosophila) |
| 36975_at | hypothetical protein MGC8721 |
| 33811_at | phosphatidylinositol glycan, class B |
| 36650_at | cyclin D2 |
| 41544_at | polo-like kinase 2 (Drosophila) |
| 36502_at | PFTAIRE protein kinase 1 |
| 34000_r_at | hypothetical protein LOC90925 |
| 40102_at | oxysterol binding protein-like 2 |
| 38068_at | autocrine motility factor receptor |
| 40375_at | early growth response 3 |
| 34365_at | peptidylprolyl isomerase E (cyclophilin E) |
| 37571_at | dedicator of cytokinesis 9 |
| 37243_at | guanylate cyclase 1, soluble, beta 3 |
| 39124_r_at | transient receptor potential cation channel, subfamily C, |
| | member 1 |
| 823_at | chemokine (C—X3—C motif) ligand 1 |
| 1530_g_at | hypothetical protein CG003 |
| 34239_at | hypothetical gene CG018 |
| 36159_s_at | prion protein (p27-30) (Creutzfeld-Jakob disease, |
| | Gerstmann-Strausler-Scheinker syndrome, fatal familial |
| | insomnia) |
| 1527_s_at | hypothetical gene CG018 |
| 37283_at | meningioma (disrupted in balanced translocation) 1 |
| 41668_r_at | TDP-glucose 4,6-dehydratase |
| 33788_at | lysosomal apyrase-like 1 |
| 39031_at | cytochrome c oxidase subunit VIIa polypeptide 1 |
| | (muscle) |
| 32531_at | gap junction protein, alpha 1, 43 kDa (connexin 43) |
| 906_at | signal transducer and activator of transcription 4 |
| 36965_at | ankyrin 3, node of Ranvier (ankyrin G) |
| 719_g_at | protease, serine, 11 (IGF binding) |
| 40961_at | SWI/SNF related, matrix associated, actin dependent |
| | regulator of chromatin, subfamily a, member 2 |
| 1597_at | growth arrest-specific 6 |
| 36118_at | nuclear receptor coactivator 1 |
| 38775_at | low density lipoprotein-related protein 1 (alpha-2- |
| | macroglobulin receptor) |
| 40834_at | PDZ domain containing 3 |
| 35221_at | purine-rich element binding protein A |
| 38113_at | spectrin repeat containing, nuclear envelope 1 |
| 38433_at | AXL receptor tyrosine kinase |
| 33804_at | PTK2B protein tyrosine kinase 2 beta |
| 32026_s_at | PDZ domain containing guanine nucleotide exchange |
| | factor (GEF) 1 |
| 39422_at | phosphodiesterase 4D interacting protein (myomegalin) |
| 1709_g_at | mitogen-activated protein kinase 10 |
| 1675_at | RAS p21 protein activator (GTPase activating protein) 1 |
| 40046_r_at | chromosome 18 open reading frame 1 |
| 36076_g_at | RAB, member of RAS oncogene family-like 4 |
| 37958_at | transmembrane 4 superfamily member 10 |
| 35794_at | ADP-ribosylation factor guanine nucleotide factor 6 |
| 36025_at | Rho guanine nucleotide exchange factor (GEF) 17 |
| 39698_at | homeodomain-only protein |
| 37723_at | cyclin G2 |
| 32542_at | four and a half LIM domains 1 |
| 41120_at | aminomethyltransferase (glycine cleavage system protein |
| | T) |
| 1529_at | hypothetical protein CG003 |
| 41620_at | dedicator of cytokinesis 4 |
| 35785_at | GABA(A) receptor-associated protein like 1 |
| 35681_r_at | zinc finger homeobox 1b |
| 36342_r_at | H factor (complement)-like 3 |
| 38705_at | ubiquitin-conjugating enzyme E2D 2 (UBC4/5 homolog, |
| | yeast) |
| 41671_at | echinoderm microtubule associated protein like 1 |
| 36935_at | RAS p21 protein activator (GTPase activating protein) 1 |
| 225_at | guanine nucleotide binding protein (G protein), beta |
| | polypeptide 3 |
| 35181_at | hypothetical protein FLJ10849 |
| 32854_at | F-box and WD-40 domain protein 1B |
| 587_at | endothelial differentiation, sphingolipid G-protein- |
| | coupled receptor, 1 |
| 41179_at | ring finger protein 44 |

The present inventors hypothesized that increased expression of certain genes in primary NSCLC could identify patients at high risk for the development of brain metastasis. Univariate Cox regression analysis of the normalized RQ-PCR values was performed (see Materials and Methods). The genes were ranked according to their effect on brain metastasis risk (Table 8, herein below).

TABLE 8

| | | | | | |
|---|---|---|---|---|---|
| gene | B | SE | Wald | df | Sig |

UNIVARIATE COX REGRESSION

| | | | | | |
|---|---|---|---|---|---|
| KIFC1 | 0.33886 | 0.174008 | 3.79 | 1 | 0.051 |
| KIF2C | 0.2585 | 0.193871 | 1.78 | 1 | 0.18 |
| KIF14 | 0.232759 | 0.190735 | 1.49 | 1 | 0.22 |
| CCNB2 | 0.224726 | 0.181883 | 1.53 | 1 | 0.22 |
| SIL | 0.27908 | 0.172756 | 2.61 | 1 | 0.106212 |
| TNPO1 | −0.26433 | 0.189937 | 1.93 | 1 | 0.16 |
| LMNB1 | −0.022765 | 0.169886 | 0.02 | 1 | 0.89 |
| CDH2 | 0.361766 | 0.162251 | 4.97 | 1 | 0.025 |
| FALZ | −0.407923 | 0.233316 | 3.06 | 1 | 0.08 |
| SGNE1 | 0.119528 | 0.163295 | 0.54 | 1 | 0.46 |
| ADAM8 | −0.077522 | 0.163499 | 0.22 | 1 | 0.64 |
| SPP1 | 0.343328 | 0.19089 | 3.23 | 1 | 0.072 |

MULTIVARIATE COX REGRESSION

| | | | | | |
|---|---|---|---|---|---|
| CDH2 | 0.382 | 0.147 | 6.766 | 1 | 0.009 |
| FALZ | −0.586 | 0.229 | 6.541 | 1 | 0.011 |
| KIFC1 | 0.475 | 0.207 | 5.297 | 1 | 0.021 |

B—regression coefficient, SE—standard error, WALD—Wald significance score, df—degrees of freedom

FIG. 3 schematically illustrates the relative effect of each gene on the risk for brain metastasis based on its Wald significance score. Genes in the center had no significant effect while genes on the right had a positive effect and genes on the left had a negative effect. A score of 2.71 and above was considered significant. Three genes had a significant (Wald score >2.71, p-value<0.1) positive predictive value, CDH2, KIFC1 and SPP1, with the SIL gene just below 2.71. Surprisingly, one gene, FALZ, had a borderline significant negative predictive value (−3.06).

Multivariate Cox regression (Table 8, herein above) analysis revealed a statistically significant positive predictive effect for CDH2 and KIFC1 (p=0.009, p=0.021) and a statistically significant negative effect for FALZ (p=0.011).

The multivariate Cox analysis was then used to define a Brain Metastasis Score, given by the following equation:

Score=0.382*N_CDH2−0.586*N_FALZ+0.475*N_KIFC1,

Where N=normalization coefficient. The patients were then ranked into 3 groups based on their score. A score of less than −0.5 was ranked 1 (low), a score between −0.5 and 0.5 was ranked 2 (intermediate), and a score above 0.5 was ranked 3 (high). These divisions were chosen to achieve approximately equal numbers of patients in each group.

Brain metastasis developed in 3 (6.7%) of the 45 patients in the low ranking (1) group, in 11 (20%) of the 54 intermediate ranked (2) patients and in 17 (41%) of the 41 patients classified to the highest rank (3; Table 9, herein below). Thus, the higher the score—the higher risk for brain metastasis.

TABLE 9

| | | |
|---|---|---|
| | | CENSORED |
| NO. OF | NO. OF | CASES |

| RANK SCORE | PATIENTS | EVENTS | No. | % |
|---|---|---|---|---|
| 1 | 45 | 3 | 42 | 93.3 |
| 2 | 54 | 11 | 43 | 79.6 |
| 3 | 41 | 17 | 24 | 58.5 |
| total | 140* | 31 | 109 | 77.9 |

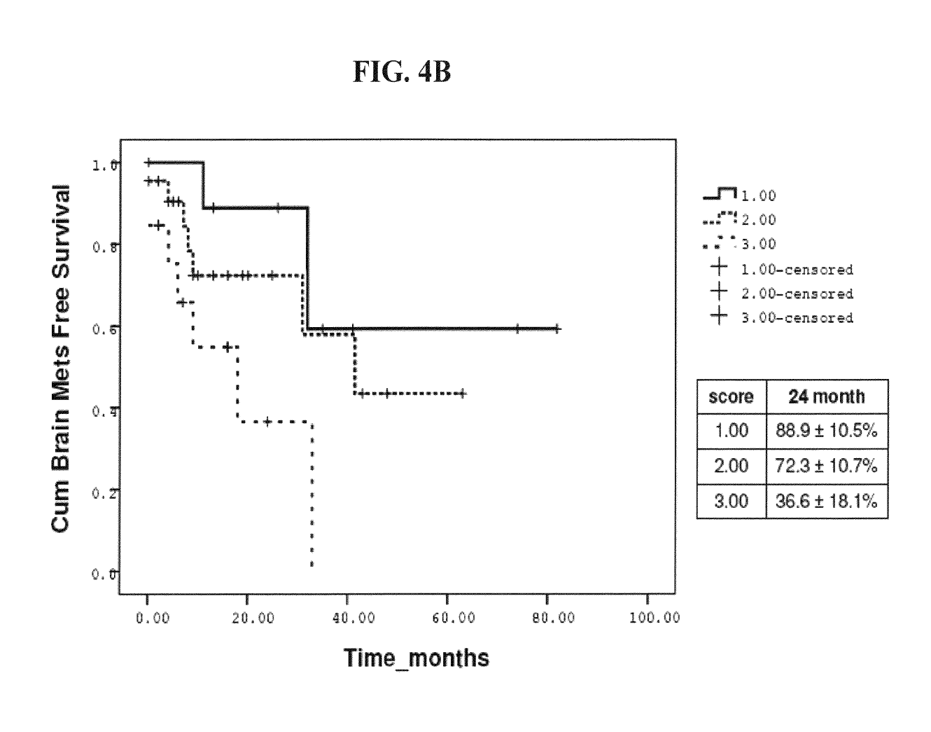

The clinical significance predicting brain metastasis in patients with early stage disease who may benefit from prophylactic therapy is of great clinical importance. The present inventors therefore calculated the score separately for the group of patients with early stage (I-II) disease and for the group with advanced stage (III-IV) disease. The results are depicted by Kaplan-Meier curves in FIGS. 4A-B. Patients with low stage NSCLC who had a high score had approximately 40% incidence of brain metastasis within the first two years after diagnosis compared with a 10% risk for patients in the low and intermediate groups (p<0.02 log rank test, FIG. 4A). The scores remained significant also in patients with more advanced lung cancer (FIG. 4B). The brain metastasis free survival at 24 months was 89% for patients with low score compared with only 37% in patients with high score (P<0.02, log rank test). Thus the combined score based on the gene expression level of all three genes in primary NSCLC is a powerful predictor of the risk for brain metastasis.

### Example 2

### Immunohistochemistry Studies

### Materials and Methods

Tumor Specimens: Tumor sections were taken from 107 formalin fixed, paraffin embedded NSCLC tumor specimens with known clinical outcomes (26 with known brain metastases, and 81 without known brain metastases). 44 samples were from tumors already analyzed by RQ-PCR and 63 were from additional, independent cases (see Table 1, herein above).

Immunostaining Procedure: Immunostaining was performed on 4 mm thick sections. Antigen was detected with a labeled Avidin-Biotin (LAB) method (Zymed Laboratories, USA); Monoclonal mouse anti-human antibody (DakoCytomation, California, USA) for CDH2 diluted 1:20 was used. A malignant mesothelioma tumor sample with high CDH2 expression served as control. All of the immunostained sections were examined independently by two pathologists (MP and EO) blinded to clinical outcomes. Immunostaining scoring was determined by estimation of the percentage of immunoreactive tumor cells in each section reviewed. Only those tumor cells showing both cytoplasmatic and strong membranous staining were considered positive. Cases showing up to 2% immunoreactive tumor cells were assigned a negative score, while cases with 2% and above immunoreactive tumor cells were assigned a positive score.

Results

Of the three genes identified in the multivariate Cox regression model as predictors of brain metastasis only N-cadherin expression can reliably be detected by immunohistochemistry on paraffin embedded tissues with commercially available antibodies. The present inventors therefore attempted to corroborate their RQ-PCR findings immunohistochemically on 107 samples, 63 of which were independent, i.e were not among the 142 samples evaluated previously by RQ-PCR.

FIGS. 5A-C depict examples of N-cadherin stains. In most of the samples the staining was focal ranging from 2-80% of the cells and varied in different areas of the tumor section. As mentioned previously, only those tumor cells showing both cytoplasmatic and strong membranous staining were considered positive. Of the 39 sections scored as positive. 14 were positive in 2-25% of the tumor cells, 13 were positive in 25-50% of the cells, and 12 were positive in more than 50% of the tumor cells within the sections.

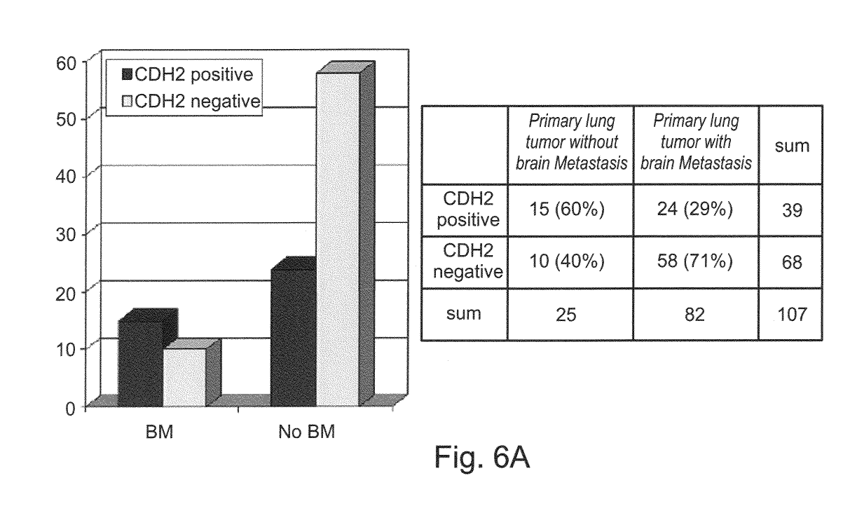

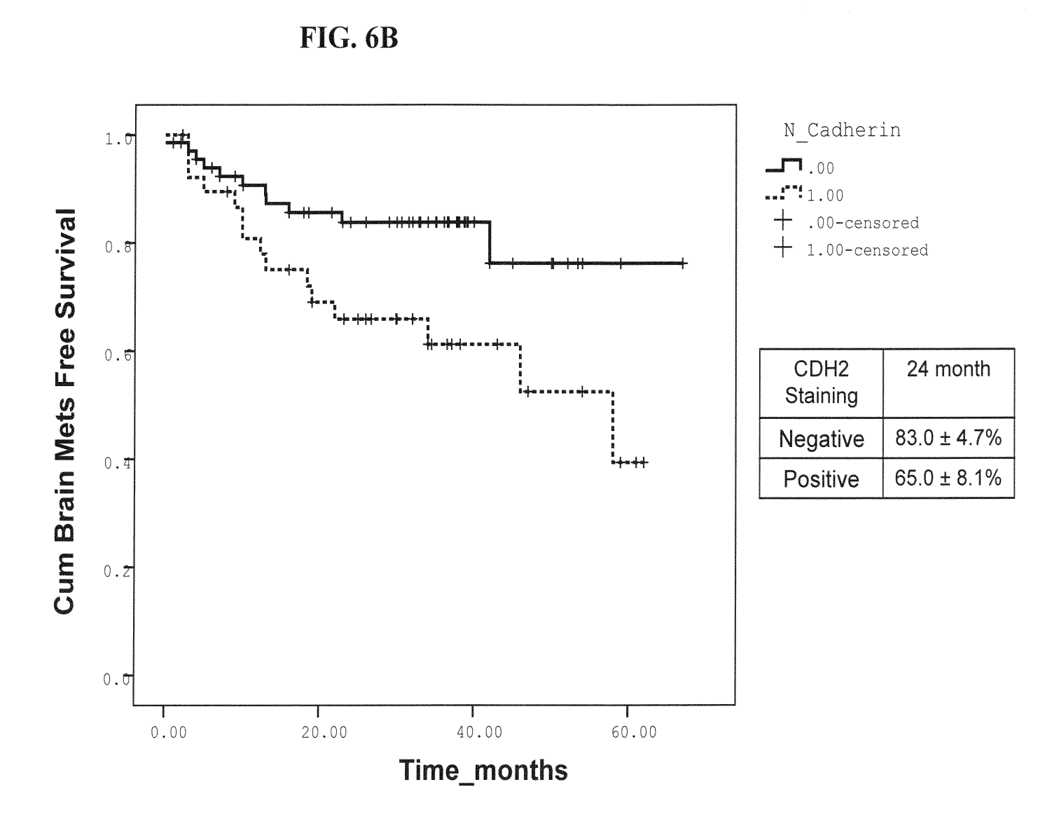

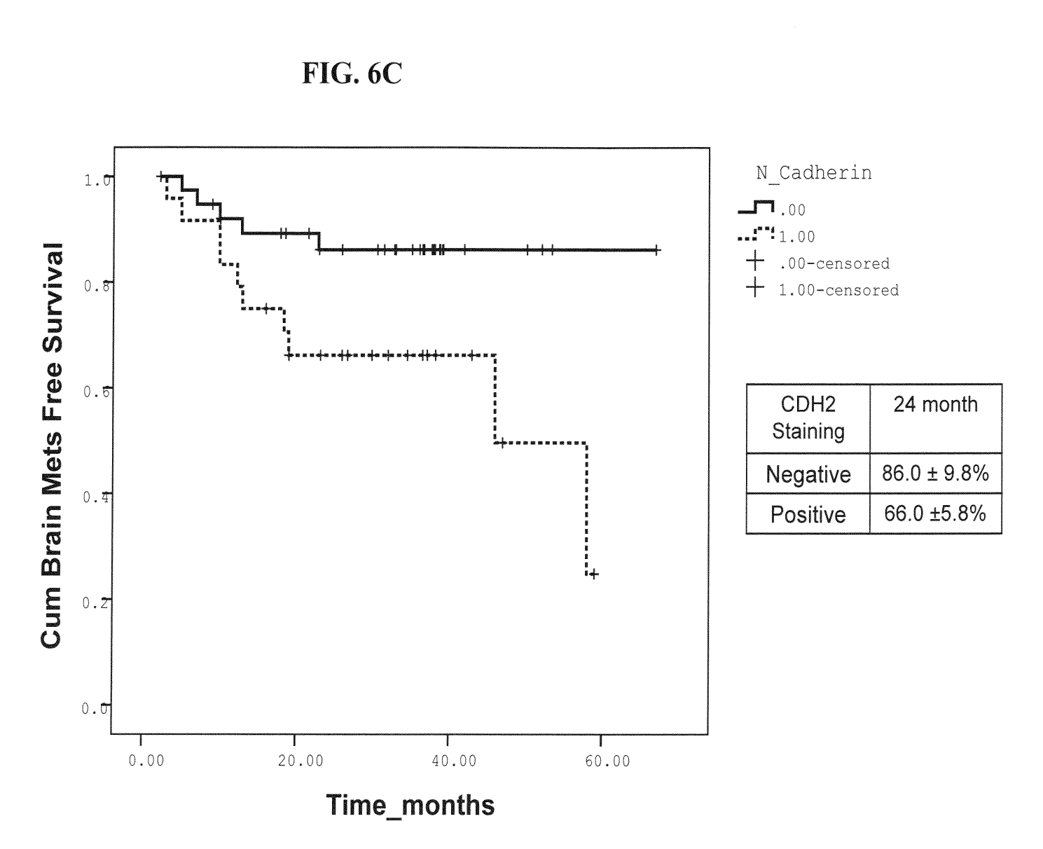

60% of the tumor samples from patients who developed brain metastasis were positive for N-cadherin compared with only 29% of the tumor samples from patients who did not develop brain metastasis. The cumulative incidence of brain metastasis in CDH2 positive cases at 24 months after diagnosis was twice as high as that seen in N-cadherin negative samples (35% vs 17%, p=0.022 log rank test) (FIGS. 6A-B). Similarly, the cumulative incidence of brain metastasis of early stage (I+II) NSCLC (N=76) patients was almost twice as high in CDH2 positive cases as that seen in N-cadherin negative samples (17% vs 10% at 24 months, p=0.079, log rank test).

Separate analysis of the 63 independent samples showed that the brain metastasis free survival at 24 months was 86% for patients with N-cadherin negative tumors compared to only 66% in positive cases (FIG. 6C, p<0.03, log rank test). Thus, while results were less robust than those for the combined RNA expression score, these immunohistochemical results for N-cadherin confirm its importance as a predictor of brain metastasis in primary NSCLC.

### DISCUSSION

About 25% of patients with NSCLC will develop brain metastases. The 3-gene model proposed herein, based on a multivariate cox regression analysis of the expression levels of 12 genes in primary NSCLC tumors, identifies a group of patients with high risk for developing of brain metastasis during the first 2 years after surgery. It was also shown that immunohistochemical detection of the expression of one of these genes, N-Cadherin, may also predict brain metastasis.

Cadherins are transmembrane proteins that mediate cell to cell adherence. They have extracellular calcium dependent domains and cytoplasmic tails that activates several signaling pathway, most notably the Wnt Beta Catenin pathway. The expression of N-cadherin has been linked to invasion and metastasis of several types of cancers [Qi J, et al., Mol Biol Cell, 2005; Asano K, et al. J Neurooncol 70:3-15, 2004; Hulit J, et al. Cancer Res 67:3106-16, 2007; Hazan R B, et al., J Cell Biol 148:779-90, 2000]. During the process of activation its extracellular domain is cleaved by ADAM10 and released to the cytosol. This is consistent with the immunohistochemical staining pattern observed, namely a membranous staining pattern with or without cytoplasmic staining. Although N-cadherin is expressed in many tissues it is highly expressed in the brain and is critical for many aspects of neuronal development through interactions with neural growth factors. It is tempting to speculate that N-Cadherin may mediate the endurance of brain metastases through interactions with the neuronal parenchyma, as observed in FIGS. 7A-E.

In multivariate COX regression analysis, KIFC1 came second after CDH2 in the association with brain metastasis. KIFC1 is one of three kinesin family proteins and is one of the five mitotic regulators that was included in the panel of genes tested. Increased expression of such mitotic spindle checkpoint genes including Aurora B kinase, MAD2, Survivin and others, have been noted associated with progression and metastasis of many types of cancers. Accordingly, novel anti mitotic and specifically kinesin-related drugs are being developed and introduced into the clinic. While KIF2C and KIF14 have been previously reported to be associated with progression of lung and breast cancers, KIFC1 has never been associated with cancer. It is unclear whether KIFC1 has a unique role in promoting the dissemination of NSCLC (into the brain) or if it simply represents the kinesin family or mitotic checkpoint proteins. Combining the expression of all the three kinesins or all the five mitotic regulators into our statistical model did improve the predictive power for brain metastasis compared with inclusion of KIFC1 alone (data not shown). While it is impossible to exclude the possibility that other mitotic regulators may have a similar or even better predictive power, in the present cohort, KIFC1 seems to be the strongest predictor of brain metastasis.

Surprisingly, the neuronal transcriptional factor FALZ (also called BPTF for bromodomain PHD finger transcription factor) was found to be a negative predictor of brain metastasis in the present cohort. FALZ was first identified by a monoclonal antibody which recognizes neurofibrillary pathology associated with Alzheimer disease and subplate neurons in the developing human brain. Except for one publication in which its overexpression in primary adenocarcinomas was predictive of metastasis, there is no data linking FALZ to cancer [Ramaswamy S, et al. Nat Genet 33:49-54, 2003].

Prophylactic CNS directed therapy has revolutionized the outcome of childhood acute lymphoblastic leukemia (ALL). Currently the intensity of the prophylactic therapy in ALL is adjusted to clinical parameters predicting the risk for CNS relapse. Prophylactic CNS irradiation (PCI) has long been accepted as a standard treatment for limited disease in small cell lung cancer where improved survival is achieved albeit at the cost of some CNS toxicity from the radiation. A prospective phase 3 study has demonstrated that PCI given to patients with extensive small cell lung cancer responding to systemic chemotherapy reduced the 1 year occurrence of brain metastasis from 40% to 13% thus extending the indication for PCI to include all patients with small cell lung cancer who respond to chemotherapy The incidence of CNS metastases in NSCLC is lower and there has therefore been reluctance to test the hypothesis of PCI with its associated CNS toxicity in these patients. A recently published study showed that PCI reduced the occurrence of brain metastases at 5 years in patients with operable stage IIIa lung cancer from 34.7% to 7.8% [Pottgen C et al., J Clin Oncol 25:4987-92, 2007] suggesting the potential benefit of PCI to a selected group of patients with NSCLC.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of predicting central nervous system (CNS) metastasis of a non-neuronal cancer in a subject, the method comprising determining a level and/or activity of N-cadherin (CDH2), in a sample of the subject wherein an increase in said CDH2 with respect to an unaffected sample is indicative of the CNS metastasis of the non-neural cancer.

2. A method of treating a subject having a non-neuronal cancer, the method comprising:
(a) determining a level and/or activity of N-cadherin (CDH2), in a sample of the subject; and
(b) determining a treatment regimen based on said level and/or activity of said CDH2.

3. The method of claim 1, further comprising determining a level and/or activity or kinesin family member C1 (KIFC1) and/or Fetal Alzheimer Antigen (FALZ1) in the sample of the subject wherein an increase in said KIFC1 and a decrease in said FALZ1 with respect to an unaffected sample is further indicative of the CNS metastasis of the non-neural cancer.

4. The method of claim 1, wherein said non-neuronal cancer is selected from the group consisting of non-small cell lung cancer, breast cancer and colon cancer.

5. The method of claim 4, wherein said non-neuronal cancer is non-small cell lung cancer.

6. The method of claim 1, further comprising determining a level and or activity of at least one additional marker involved in cell proliferation and mitosis, wherein an increase in said additional marker is further indicative of CNS metastasis of the neuronal cancer.

7. The method of claim 6, wherein said at least one additional marker is selected from the group consisting of KIFC1 (kinesin family member C1), KIF2C (kinesin family member 2C), KIF14 (kinesin family member 14), CCNB2 (cyclin B2), SIL (SCL-TAL1 interrupting locus) and TNPO1 (transportin I).

8. The method of claim 2, wherein said treatment regimen is selected from the group consisting of CNS radiotherapy, intrathecal chemotherapy and intravenous chemotherapy.

9. A kit for predicting CNS metastasis of a non-neuronal cancer in a subject, the kit comprising a packaging material which comprises at least one agent for specifically determining a level and/or activity of no more than one hundred markers, wherein at least one of said one hundred markers is N-cadherin (CDH2).

10. The kit of claim 9, wherein the kit further comprises agents for specifically determining a level and/or activity of at least one marker selected from the group consisting of kinesin family member C1 (KIFC1) and Fetal Alzheimer Antigen (FALZ1).

11. The kit of claim 9, wherein the kit further comprises agents for specifically determining a level and/or activity of at least one marker selected from the group consisting of KIF2C (kinesin family member 2C), KIF14 (kinesin family member 14), CCNB2 (cyclin B2), SIL (SCL-TAL1 interrupting locus) and TNPO1 (transportin I).

12. The method of claim 2, further comprising determining a level and/or activity or kinesin family member C1 (KIFC1) and/or Fetal Alzheimer Antigen (FALZ1) in the sample of the subject wherein an increase in said KIFC1 and a decrease in said FALZ1 with respect to an unaffected sample is further indicative of the CNS metastasis of the non-neural cancer.

13. The method of claim 2, wherein said non-neuronal cancer is selected from the group consisting of non-small cell lung cancer, breast cancer and colon cancer.

14. The method of claim 2, further comprising determining a level and or activity of at least one additional marker involved in cell proliferation and mitosis, wherein an increase in said additional marker is further indicative of CNS metastasis of the neuronal cancer.
